(19) United States
(12) Patent Application Publication
(10) Pub. No.: US 2004/0157289 A1
(43) Pub. Date: Aug. 12, 2004

(54) Protein expression system

(76) Inventors: John C. Salerno, Averill Park, NY (US); Michael Hanna, Averill Park, NY (US); Jane F. Koretz, Slingerlands, NY (US); Donna Crone, Troy, NY (US); Susan M. E. Smith, Averill Park, NY (US)

Correspondence Address: DARBY & DARBY P.C., P. O. BOX 5257, NEW YORK, NY, 10150-5257, US

(21) Appl. No.: 10/657,740

(22) Filed: Sep. 8, 2003

Related U.S. Application Data
(60) Provisional application No. 60/408,680, filed on Sep. 6, 2002.

(51) Int. Cl. C07K 14/47; C12Q 1/68; C07H 21/04

(52) U.S. Cl. 435/69.1; 530/350; 435/320.1; 435/325; 536/23.5; 435/6

(57) ABSTRACT

The present invention relates to a novel protein expression system having an oligonucleotide encoding a small heat shock protein (sHSP) operably linked to a promoter and an oligonucleotide encoding a protein of interest. In one embodiment the expressed sHSP is a truncated α-crystallin polypeptide derived from a wild-type α-crystallin protein, wherein the truncated sHSP lacks an N-terminal sequence present in the wild-type α-crystallin polypeptide. In an additional embodiment, a protein is coexpressed with a sHSP, thereby increasing the level of expression, enhancing folding and increasing the solubility of the protein.

This patent application claims the priority of U.S. provisional patent application No. 60/408,680, filed Sep. 6, 2002, which is incorporated herein by reference.

### FIELD OF THE INVENTION

The present invention relates to a novel protein expression system containing an oligonucleotide encoding a small heat shock protein operably linked to a promoter and an oligonucleotide sequence encoding a protein of interest. This protein expression system may be used to enhance protein expression and to prevent protein aggregation. Also provided is a novel truncated α-crystallin polypeptide and a chimeric protein including the same.

### BACKGROUND OF THE INVENTION

### Molecular Chaperones/Chaperonins

Chaperones are cytoplasmic proteins found in prokaryotes and eukaryotes that bind to nascent or unfolded polypeptides and ensure correct folding or transport. Chaperone proteins do not covalently bind to their targets and do not form part of the finished product. Heat-shock proteins are an important subset of the chaperone family of proteins. Molecular chaperones are currently classified into eight different families: small heat shock proteins (sHSPs); hsp60; hsp70; hsp90; hsp100; calnexin and calreticulin; folding catalysts; and prosequences. Beyond these major families are other proteins with similar functions, including nucleoplasmin, secB, and T-cell receptor associated proteins. Studies indicate that many chaperones are dependent upon hydrolysis of adenine triphosphate (ATP) for activity.

Chaperonins are a class of sequence-related molecular chaperones found in bacteria, mitochondria, and plastids. Chaperonins are abundant constitutive proteins that increase in amount upon exposure to certain stresses, such as heat shock, bacterial infection of macrophages, and an increase in the cellular content of unfolded proteins. Bacterial chaperonins are major immunogens in human bacterial infections because of their accumulation during the stress of infection. Two members of this class of chaperones are chaperonin 10 (groES; hsp10) and chaperonin 60.

### Heat Shock Proteins

Heat shock proteins (HSPs) are induced in many cells at high temperatures and contribute to the viability of cells under temperature stress. Many of these proteins are molecular chaperonins that help other proteins fold correctly and may also contribute to their stability, particularly at high temperatures. Five classes of HSPs act as molecular chaperones to prevent the misfolding of proteins. Hsp100, hsp90, hsp70, and hsp60 are large, multidomain structures, while sHSPs are much smaller, ranging in molecular weight from 12-40 kD. Examples of sHSPs include plant hsp11 and hsp12, animal hsp27, and crystallins.

The sHSP superfamily of proteins are distinct from other molecular chaperones, such as groEL and groES. For example, other molecular chaperones, particularly those that utilize ATP may cause poor growth cells if over-expressed, whereas over-expression of sHSPs is not harmful to cells. In addition, this superfamily of proteins share unique structural elements not observed in other molecular chaperones. For example, sHSPs share approximately 20% sequence identity, they generally contain at least seven β-sheets organized in a compact tertiary structure, and they share a conserved Pro-Lys repeat region at the C-terminus. Moreover, sHSPs commonly form aggregates, although the size and organization of these aggregates vary. Finally, unlike groEL and groES, sHSPs do not use ATP for chaperone activity.

Many proteins require one or more chaperonins to fold correctly in their natural expression system. An example is the photosynthetic enzyme, ribulose bis-phosphate carboxylase, which requires two chaperonins equivalent to the E. coli chaperonins groEL and groES. Several patents have been issued for methods using chaperonins to enhance the expression of native folded proteins. Some of these use different variants of the large chaperonin superfamily, such as hsp60 and hsp70. For example, U.S. Pat. No. 5,552,301 to Baneyx et al. (“Baneyx”) describes a process for enhanced production of foreign proteins in a biologically active form in bacteria by transforming a vector encoding a foreign gene into an E. coli strain which contains a mutation that results in increased production of the sigma-32 RNA polymerase subunit. As a result, the concentration of heat shock proteins in the cell is increased and culturing the transformed host at various temperatures and for various time periods leads to enhanced protein expression as compared to wild-type transformants.

U.S. Pat. No. 5,919,682 to Masters et al. (“Masters”) describes a method of overproducing functional nitric acid synthase in a prokaryote using a pCW vector under the control of tac promoter and co-expressing the protein with chaperonins. The chaperonins used to enhance expression in Masters are hsp6, hsp10, hsp90, groEL, groES, and CCT (TCP-1 complex).

U.S. Pat. No. 5,773,245 to Wittrup et al. (“Wittrup”) describes methods of increasing secretion of an overexpressed gene product in a host cell by inducing expression of chaperone proteins within the cell. The chaperones used in Wittrup include the hsp70 family of protein, such as mammalian or yeast, hsp68, hsp72, hsp73, clathrin uncoating ATPase, IgG heavy chain binding protein (BiP), glucose-regulated proteins 75, 78 and 80 (GRP75, GRP78, GRP80, respectively), HSC70, and yeast KARz, BiP, SSA1-4, SSB1, SSD1, and the like.

U.S. Pat. No. 5,561,221 to Yoshida et al. (“Yoshida”) relates to monomeric subunits of chaperonin-60 or truncated fragments thereof that promote protein folding in vitro. Yoshida states that monomeric subunits of chaperonin-60 or fragments of an unfolded polypeptide from an inactive conformation.

Finally, U.S. Pat. No. 4,758,512 to Goldberg et al. (“Goldberg”) relates to the production of host cells having specific mutations within their DNA sequences which cause the organism to exhibit a reduced capacity for degrading foreign products. These mutated host organisms can be used to increase yields of genetically engineered foreign proteins. In particular, Goldberg contemplates producing a polypeptide in a host that carries a mutation in a heat shock regulatory gene so that the polypeptide remains intact when it is expressed in the host.

### α-Crystallins

In addition to the groEL/groES superfamily of proteins, a completely unrelated superfamily of sHSPs exists: α-crystallins. α-crystallins are associated with a variety of tissues and physiological functions. One isoform, αB-crystallin, is more commonly involved in both normal and pathological processes than the second αA isoform (Bhat, et al., Biochem. Biophys. Acta., 158:319-325, 1989). The two α-crystallin isoforms are heavily co-expressed only in the mammalian lens, where the very high concentration of these coaggregates in the cell cytoplasm provides the extra refractive power needed by the visual system for focus on the retina. The lens α-crystallins are notable for their long-term stability, which allows them to exist essentially intact for an organism's life in the metabolically inactive lens interior. They are also known for their unusual aggregation properties, which enable them to maintain lens transparency without significant scattering in the visible region of the electromagnetic spectrum.

α-crystallins are homologous to sHSPs (Ingola, et al., Proc. Natl. Acad. Sci. U.S.A., 79(7):2360-2364, 1989) and have chaperone-like activity under some conditions. α-crystallin has been shown to prevent protein aggregation and to promote protein folding, particularly at elevated temperatures (Horwitz, J., Proc. Natl. Acad. Sci. U.S.A., 89(21):10449-10453, 1992). Properties that allow sHSPs to stabilize folding intermediates may contribute to the stability of α-crystallins (Doss-Pepe, et al., Exp. Eye Res., 67(6):657-679, 1998), and may allow them to stabilize other lens components.

The ability of these proteins to form relatively large self-limiting structures without a high degree of order is crucial in determining their suitability as refraction-enhancing solute particles in the lens. Several models for α-crystallin aggregate structures have been proposed (Seizen, et al., Eur. J. Biochem., 111(2):435-444, 1980; Wisow, Exp. Eye Res. 56(6):729-732, 1993; Tardieu, et al., J. Mol. Biol., 192(4):711-724, 1986), but the one most consistent with the protein's solution properties and physiological constraints is the micellar protein model first proposed by Augusteyn and Koretz (FEBS Lett., 22(1):1-5, 1987). This model, which assumes that α-crystallin aggregation is characterized primarily by non-specific hydrophobic interactions, is consistent with the primary sequence's hydropathy profile, polydispersity in solution, reported interactions with detergents, association with membranes, occupation of equivalent microenvironments in the oligomer, as well as other factors suggesting that the α-crystallin subunit is amphipathic (Augusteyn, et al., Biochim. Biophys. Acta., 915(1):132-139, 1987). More recently, it has been shown that aggregates prepared from recombinant α-crystallin form polydisperse hollow spheres and ellipsoids with structural and solution properties very similar to those of crystallins expressed in mammalian lenses (Haley, et al., J. Mol. Biol., 277(1):27-35, 1998).

Considerable regions of hydrophobic sequence are present in α-crystallins, and speculation has naturally arisen concerning the nature of the exposed hydrostatic patches. There are three exons in the structural gene encoding each of the two α-crystallin isoforms αA and αB (van den Heuvel, et al., J. Mol. Biol., 185(2):273-284, 1985), and the prevailing model has been of a two domain structure, with the N-terminal region providing the more hydrophobic surfaces (Carver, et al., Biochim. Biophys. Acta., 116(1):22-28, 1993). Some have proposed two sheet domains linked by an extended hydrophobic loop (Fransworth, et al., Int. J. Biol. Macromol., 22(3-4):175-85, 1998), since both secondary structural modeling and circular dichroism studies indicate that α-crystallin is primarily a β-sheet structure (Koretz, et al., Int. J. Biol. Macromol., 22 (3-4):283-294, 1998).

Recently, Kim et al. reported the first crystal structure of a sHSP, MjHSP16.5, providing long-awaited insight into the common structural features of the superfamily. The structure consists of a spherical twenty four subunit aggregate (Nature, 394(6693):595-599, 1998). The building block of the sphere is dimeric, with two monomers, consisting of two antiparallel β sheets each per dimer. Each monomer contributes a single β strand to the N terminal edge of one sheet of the other monomer. This provides a mechanism for dimer formation, while suggesting that the tertiary structure is greatly stabilized by dimerization. Homology between other sHSPs and α-crystallin extends over a large number of families and bridges kingdom boundaries; the superfamily is evidently both ancient and widespread (de Jong, et al., Int. J. Biol. Macromol., 22(3-4):151-162, 1988).

However, to date, no one has identified those regions of sHSPs, or in particular, α-crystallins, that are critical to their chaperonin activity, nor has anyone exploited the unique abilities of sHSPs and α-crystallins to enhance protein expression and to facilitate protein folding.

### SUMMARY OF THE INVENTION

The present invention provides a method of enhancing the expression and/or secretion of proteins or polypeptides by coexpressing the protein or polypeptide with a small heat shock protein in a host. In a preferred embodiment, the sHSP used in the method of the present invention is a truncated α-crystallin polypeptide derived from a wild-type α-crystallin protein (SEQ ID NO: 1), wherein the truncated polypeptide lacks an N-terminal sequence present in the wild-type protein. In a further preferred embodiment, the N-terminal sequence of the wild-type protein that is eliminated from the truncated form is hydrophobic and it precedes a common domain in the wild-type protein. Preferably, the truncated α-crystallin polypeptide lacks the N-terminal sequence of the wild-type protein that includes residues 1-51, as set forth in SEQ ID NO: 3. In another embodiment, the wild-type protein as set forth in SEQ ID NO: 1, may be truncated between residues 52 and 55 resulting in a truncated α-crystallin polypeptide having between 122 and 119 amino acid residues.

The present invention also provides an isolated polypeptide including an amino acid sequence encoded by a nucleic acid that hybridizes, under stringent conditions, to the complement of a nucleic acid encoding the polypeptide described above. This polypeptide is optionally at least 70% identical to a polypeptide comprising the amino acid sequence set forth in SEQ ID NO: 1 (FIG. 1). Alternatively, the polypeptide described above has an amino acid sequence at least 80% identical to the amino acid sequence of the polypeptide sequence set forth in SEQ ID NO: 1 (FIG. 1) using a BLAST algorithm. Preferably, the polypeptide has an amino acid sequence more than 90% identical to the amino acid sequence of the polypeptide sequence set forth in SEQ ID NO: 1 (FIG. 1) using a BLAST algorithm.

In an alternative embodiment of the present invention, the polypeptide described above optionally includes a linker sequence at the N-terminus which is designed to enhance the solubility of the polypeptide.

Also provided is an isolated nucleic acid encoding the truncated α-crystallin polypeptide described above, as well as an isolated nucleic acid that hybridizes, under stringent conditions, to the complement of a nucleic acid encoding the polypeptide described above, as set forth in SEQ ID NO: 2 (FIG. 2).

The present invention further provides an expression vector including a nucleic acid encoding a sHSP, and a nucleic acid encoding a protein, polypeptide, or fragment thereof, wherein the nucleic acids are operatively associated with an expression control sequence. The sHSP encoded by a nucleic acid sequence contained with the expression vector described above is preferably selected from the group consisting of a wild-type α-crystallin protein; a truncated α-crystallin polypeptide; a thermophilic sHSP; a chimeric polypeptide including (a) a wild-type α-crystallin protein or a truncated α-crystallin polypeptide and (b) thermophilic sHSP; (c) or combinations thereof. In a more preferred embodiment, the sHSP is a chimeric polypeptide including a truncated α-crystallin polypeptide and thermophilic sHSP. Preferably, the truncated α-crystallin polypeptide lacks an N-terminal sequence present in a wild-type α-crystallin protein, and that sequence is hydrophobic and precedes a common domain in the wild-type protein.

In a most preferred embodiment of the present invention, the expression vector contains a nucleic acid sequence encoding a truncated α-crystallin polypeptide lacking an N-terminal sequence that comprises residues 1-51 of the corresponding wild-type protein, as set forth in SEQ ID NO: 2 (FIG. 2).

In addition, the present invention provides a method of enhancing expression and/or secretion of a protein in a host cell that includes coexpressing the protein with a sHSP. The sHSP is preferably selected from the group consisting of a wild-type α-crystallin protein; a truncated α-crystallin polypeptide; thermophilic sHSP; a chimeric polypeptide including (a) a wild-type α-crystallin protein or a truncated α-crystallin polypeptide and (b) thermophilic sHSP; or (c) combinations thereof. In a more preferred embodiment, the sHSP is a chimeric polypeptide including a truncated α-crystallin polypeptide and thermophilic sHSP. Preferably, the truncated α-crystallin polypeptide lacks an N-terminal sequence present in a wild-type α-crystallin protein, and that sequence is hydrophobic and precedes a common domain in the wild-type protein. In a most preferred embodiment of the present invention, the method of the present invention includes coexpressing a protein with a truncated a:—crystallin polypeptide lacking an N-terminal sequence that contains residues 1-51 of the corresponding wild-type protein, as set forth in SEQ ID NO: 1 (FIG. 1).

Finally, the present invention provides a thermotolerant host cell, which is capable of surviving at temperatures greater then those tolerated by a wild type cell, genetically modified to express a sHSP. The sHSP is preferably selected from the group consisting of a wild-type α-crystallin protein; a truncated α-crystallin polypeptide; thermophilic sHSP; a chimeric polypeptide including (a) a wild-type α-crystallin protein or a truncated α-crystallin polypeptide and (b) thermophilic sHSP; or (c) combinations thereof. In a more preferred embodiment, the sHSP is a chimeric polypeptide including a truncated α-crystallin polypeptide and thermophilic sHSP. Preferably, the truncated α-crystallin polypeptide lacks an N-terminal sequence present in a wild-type α-crystallin protein, and that sequence is hydrophobic and precedes a common domain in the wild-type protein. In a most preferred embodiment of the present invention, the thermotolerant host cell expresses a truncated α-crystallin polypeptide lacking an N-terminal sequence that contains residues 1-51 of the corresponding wild-type protein, as set forth in SEQ ID NO: 1 (FIG. 1).

These and other alternative non-limiting embodiments of the present invention will be described in the following description and in the attached figures.

### BRIEF DESCRIPTION OF THE DRAWINGS

FIG. 1 shows the amino acid sequence of wild type α-crystallin, GenBank Accession No. P02489 (SEQ ID NO:1)

FIG. 2 shows a nucleotide sequence which encodes a wild type α-crystallin having a truncated N-terminus (SEQ ID NO: 2).

FIG. 3 shows an amino acid sequence of wild type α-crystallin having a truncated N-terminus (SEQ ID NO: 3).

FIGS. 4A and 4B when joined at matchline A-A show the sequence alignment of representative members of the small heat shock protein superfamily (Sutton, et al., Science, 273:1058-1073, 1996; Tseng, et al., Plant Mol. Bio., 18:963-965, 1992). Sequences correspond to GenBank accession numbers 2495337 (hsp16.5; SEQ ID NO: 4), P27777 (hs11_orysa; SEQ ID NO: 5), P19243 (hs11_pea; SEQ ID NO: 6), P06582 (hs12_caee1; SEQ ID NO: 7), Q06823 (sp_{—}21_STIAU; SEQ ID NO: 8), P14602 (hs27_mouse; SEQ ID NO: 9), P02470 (craa_bovin; SEQ ID NO: 10), P02510 (crab_bovin; SEQ ID NO: 11), and P24622 (cra2_mouse; SEQ ID NO: 12). The putative disordered N terminal region shows little homology between families, while the region corresponding to the β sheet domain of sHSP16.5 is much more conserved. The sequence locations corresponding to the secondary structural features of sHSP16.5 are indicated.

FIG. 5 shows a slightly altered sequence alignment reflecting information additional to the sequences themselves (Berengian et al., Biol. Chem. 274(10):6305-6314, 1999). The orientation of HSP 16.5 secondary structural elements (SEQ ID NO: 17) relative to the α-crystallin sequences (SEQ ID NO: 18) is emphasized. Boxed regions correspond to conserved beta strands.

FIG. 6 shows a comparison of the folding topologies of small heat shock proteins (left), including the alpha-crystallins; and the immunoglobulin fold (right). Although both have cores composed of seven β strands, the topologies are fundamentally different.

FIG. 7 (A-B) shows a model structure of αA-crystallin, based on homology modeling of HSP 16.5. Only the extended core region of αA-crystallin (residues 50-145) is shown. FIG. 7A shows a ribbon structure representing the backbone topology, gray-scaled to differentiate amino acids with different properties. The loop connecting the putative short first strand with the second strand is in the foreground on the left. FIG. 7B Structure with side chains represented and critical residues labeled. Note that R116 (R120 in a β-crystallin) appears to stabilize an exposed loop and connects the two sheets which make up the core structure through H bonding. The view provided is that which would be seen by looking into the hydrophobic region between the two sheets. The extended loop on the left is a foreshortened version of the region which forms β6 in HSP 16.5. The structure of this loop is unknown, and it is displayed merely to indicate its size and position. It is likely to be involved in dimer formation.

FIG. 8 shows the results of aggregation assays used to assess the ability of the construct α-crystallin_{Δ51+} to reduce insulin aggregation.

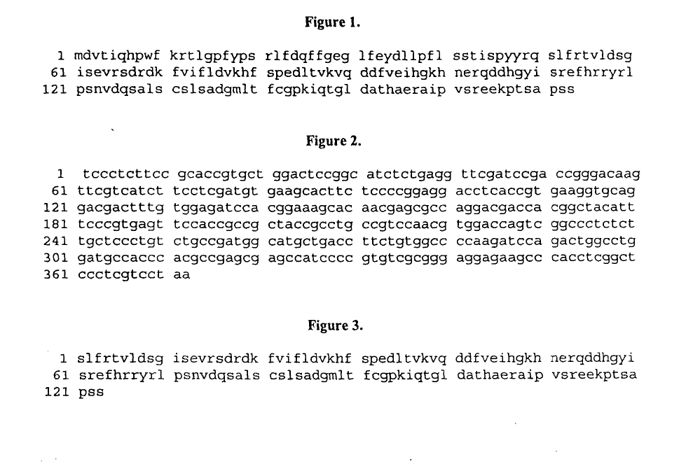

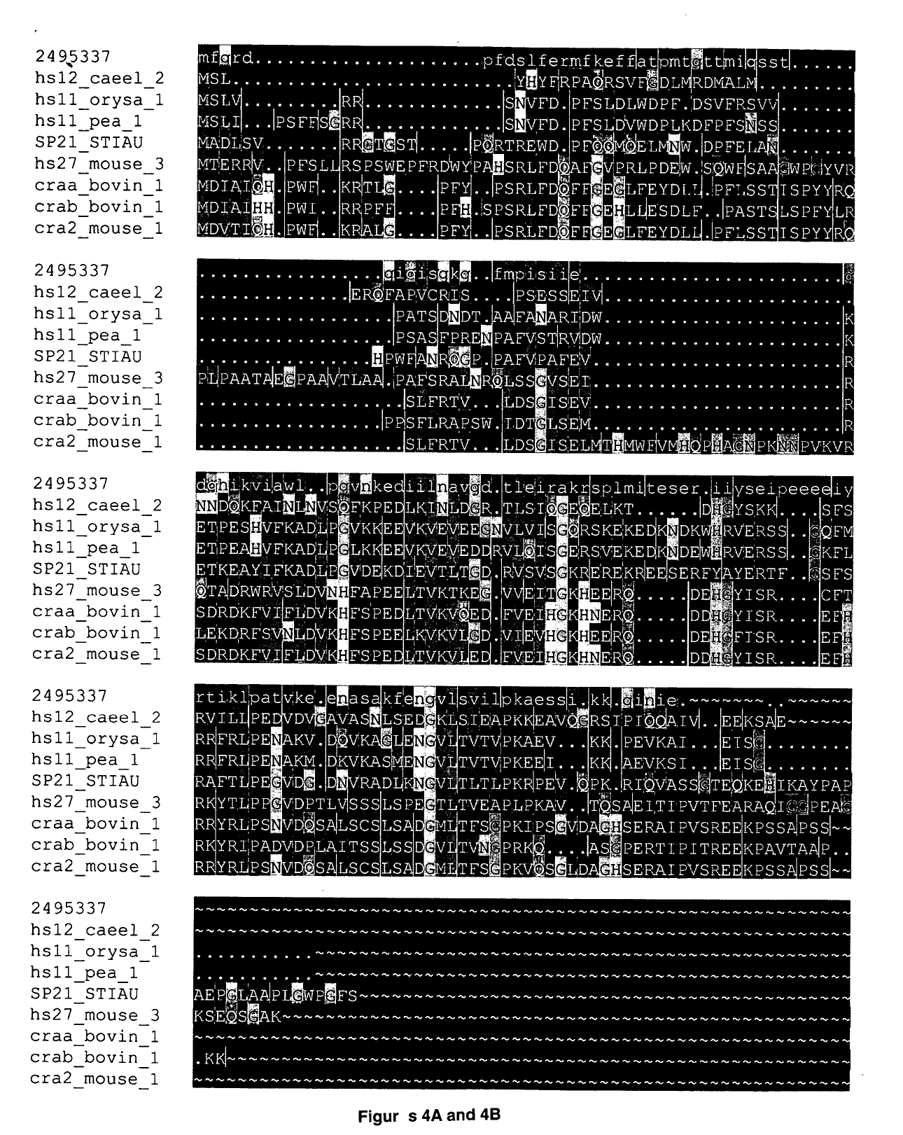

### DETAILED DESCRIPTION OF THE INVENTION

A method of enhancing the expression and/or secretion of proteins and/or polypeptides in vitro has been developed in which the protein or polypeptide is coexpressed with a sHSP. In a preferred embodiment, the sHSP includes a truncated α-crystallin polypeptide derived from a wild-type α-crystallin protein, wherein the truncated polypeptide lacks an N-terminal sequence present in the wild-type protein. It has been surprisingly found that α-crystallin is a one-domain protein, and that this domain is larger and more organized than previously thought. In addition, it has been found that the tertiary structure of α-crystallin takes the form of a highly stable sandwich that is stable against environmental stressors and site-directed mutagenesis. Investigators have reported mutagenesis directed at over thirty sites with negligible effects on stability of α-crystallin (Smulders, R. H. et al., Int. J. Biol. Macromol. 22(3-4):187-96, 1998). Most significant is the observation that the aggregation of α-crystallin is controlled by the N-terminal extension and more specifically, approximately the first 51 residues of the protein.

Before the present invention is described in more detail, the following definitions are offered as illustrations of the scope of the invention. However, these definitions should not be construed as limitations on the present invention.

### Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

### General Definitions

As used herein, the term “isolated” means that the referenced material is removed from the environment in which it is found. Thus, an isolated biological material can be free of cellular components, i.e., components of the cells in which the material is found or produced. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

Methods for purification are well-known in the art. For example, nucleic acids can be purified by precipitation, chromatography (including preparative solid phase chromatography, oligonucleotide hybridization, and triple helix chromatography), ultracentrifugation, and other means. Polypeptides and proteins can be purified by various methods including, without limitation, preparative disc-gel electrophoresis, isoelectric focusing, HPLC, reversed-phase HPLC, gel filtration, ion exchange and partition chromatography, precipitation and salting-out chromatography, extraction, and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents. Cells can be purified by various techniques, including centrifugation, matrix separation such as nylon wool separation, panning and other immunoselection techniques, depletion methods such as complement depletion of contaminating cells, and cell sorting techniques such as fluorescence activated cell sorting (FACS). Other purification methods are possible. A purified material may contain less than about 50%, preferably less than about 75%, and most preferably less than about 90%, of the cellular components with which it was originally associated. The “substantially pure” indicates the highest degree of purity which can be achieved using conventional purification techniques known in the art.

A “sample” as used herein refers to a biological material which can be tested, for the presence of wild-type proteins coexpressed with sHSPs, to identify cells that specifically express the wild-type protein. Such samples can be obtained from any source, including without limitation, prokaryotic cells and eucaryotic cells such as E. coli.

In preferred embodiments, the terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The invention also contemplates fragments of sHSPs and the uses thereof. A “fragment” preferably retains at least a portion of the biological activity of the corresponding full-length polypeptides, at least 50% activity, preferably at least 75%, and most preferably, at least 90% of a truncated α-crystallin lacking the first 51 residues of the N-terminus. Alternatively, a fragment of the invention may also exhibit enhanced activity relative to the full-length polypeptide, for example, at least twice as much, more than ten times as much, preferably more than fifty times as much, and most preferably at least 100 times the biological activity of the corresponding full-length polypeptide.

### Molecular Biology Definitions

In accordance with the present invention, there may be employed conventional molecular biology, microbiology and recombinant DNA techniques within the skill of the art. Such techniques are explained fully in the literature. See, for example, Sambrook, Fitsch & Maniatis, Molecular Cloning: A Laboratory Manual, Second Edition (1989) Cold Spring Harbor Laboratory Press, Cold Spring Harbor, N.Y. (referred to herein as “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins, eds. 1984); Animal Cell Culture (R. I. Freshney, ed. 1986); Immobilized Cells and Enzymes (IRL Press, 1986); B. E. Perbal, A Practical Guide to Molecular Cloning (1984); F. M. Ausubel et al. (eds.), Current Protocols in Molecular Biology, John Wiley & Sons, Inc. (1994).

The term “polymer” means any substance or compound that is composed of two or more building blocks (‘mers’) that are repetitively linked together. For example, a “dimer” is a compound in which two building blocks have been joined togther; a “trimer” is a compound in which three building blocks have been joined together; etc.

The term “polynucleotide” or “nucleic acid molecule” as used herein refers to a polymeric molecule having a backbone that supports bases capable of hydrogen bonding to typical polynucleotides, wherein the polymer backbone presents the bases in a manner to permit such hydrogen bonding in a specific fashion between the polymeric molecule and a typical polynucleotide such as single-stranded DNA. Such bases are typically inosine, adenosine, guanosine, cytosine, uracil and thymidine. Polymeric molecules include “double stranded” and “single stranded” DNA and RNA, as well as backbone modifications thereof (for example, methylphosphonate linkages).

Thus, a “polynucleotide” or “nucleic acid” sequence is a series of nucleotide bases (also called “nucleotides”), generally in DNA and RNA, and means any chain of two or more nucleotides. A nucleotide sequence frequently carries genetic information, including the information used by cellular machinery to make proteins and enzymes. The terms include genomic DNA, cDNA, RNA, any synthetic and genetically manipulated polynucleotide, and both sense and antisense polynucleotides.

This includes single- and double-stranded molecules; i.e., DNA-DNA, DNA-RNA, and RNA-RNA hybrids as well as “protein nucleic acids” (PNA) formed by conjugating bases to an amino acid backbone. This also includes nucleic acids containing modified bases, for example, thio-uracil, thio-guanine and fluoro-uracil.

The polynucleotides herein may be flanked by natural regulatory sequences, or may be associated with heterologous sequences, including promoters, enhancers, response elements, signal sequences, polyadenylation sequences, introns, 5′- and 3′-non-coding regions and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages such as methyl phosphonates, phosphotriesters, phosphoroamidates, carbamates, and with charged linkages such as phosphorothioates and phosphorodithioates. Polynucleotides may contain one or more additional covalently linked moieties, such as proteins such as nucleases, toxins, antibodies, signal peptides, poly-L-lysine, intercalators, chelators such as metals, radioactive metals, iron, oxidative metals and alkylators to name a few. The polynucleotides may be derivatized by formation of a methyl or ethyl phosphotriester or an alkyl phosphoramidite linkage. Furthermore, the polynucleotides herein may also be modified with a label capable of providing a detectable signal, either directly or indirectly. Exemplary labels include radioisotopes, fluorescent molecules, biotin and the like. Other non-limiting examples of modification which may be made are provided, below, in the description of the present invention.

A “polypeptide” is a chain of chemical building blocks called amino acids that are linked together by chemical bonds called “peptide bonds”. The term “protein” refers to polypeptides that contain the amino acid residues encoded by a gene or by a nucleic acid molecule such as an mRNA or a cDNA, transcribed from that gene either directly or indirectly. Optionally, a protein may lack certain amino acid residues that are encoded by a gene or by an mRNA. For example, a gene or mRNA molecule may encode a sequence of amino acid residues on the N-terminus of a protein, such as a signal sequence, that is cleaved from, and therefore may not be part of, the final protein. A protein or polypeptide, including an enzyme, maybe a “native” or “wild-type”, meaning that it occurs in nature; or it may be a “mutant”, “variant” or “modified”, meaning that it has been made, altered, derived, or is in some way different or changed from a native protein or from another mutant.

“Amplification” of a polynucleotide, as used herein, denotes the use of polymerase chain reaction (PCR) to increase the concentration of a particular DNA sequence within a mixture of DNA sequences. For a description of PCR see Saiki et al., Science, 239:487, 1988.

“Chemical sequencing” of DNA denotes methods such as that of Maxam and Gilbert (Maxam-Gilbert sequencing; see Maxam & Gilbert, Proc. Natl. Acad. Sci. U.S.A. 1977, 74:560), in which DNA is cleaved using individual base-specific reactions.

“Enzymatic sequencing” of DNA denotes methods such as that of Sanger (Sanger et al., Proc. Natl. Acad. Sci. U.S.A., 74:5463, 1977) and variations thereof well known in the art, in a single-stranded DNA is copied and randomly terminated using DNA polymerase.

A “gene” is a sequence of nucleotides which code for a functional “gene product”. Generally, a gene product is a functional protein. However, a gene product can also be another type of molecule in a cell, such as an RNA and more specifically either a tRNA or a rRNA. For the purposes of the present invention, a gene product also refers to an mRNA sequence which may be found in a cell. For example, measuring gene expression levels according to the invention may correspond to measuring mRNA levels. A gene may also comprise regulatory, non-coding, sequences as well as coding sequences. Exemplary regulatory sequences include promoter sequences, which determine, for example, the conditions under which the gene is expressed. The transcribed region of the gene may also include untranslated regions including introns, a 5′-untranslated region (5′-UTR) and a 3′-untranslated region (3′-UTR).

A “coding sequence” or a sequence “encoding” an expression product, such as a RNA, polypeptide, protein or enzyme, is a nucleotide sequence that, when expressed, results in the production of that RNA, polypeptide, protein or enzyme; i.e., the nucleotide sequence “encodes” that RNA or it encodes the amino acid sequence for that polypeptide, protein or enzyme.

An “expression control sequence” is a DNA regulatory region capable of facilitating the information in a gene or DNA sequence to become manifest, thereby producing RNA (rRNA or mRNA) or a protein by activating the cellular functions involved in transcription and translation of a corresponding gene or DNA sequence. For example, an expression control sequence may include a promoter sequence, which is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently found, for example, by mapping with nuclease S1), as well as protein binding domains (consensus sequences) responsible for the binding of RNA polymerase. The expression control sequence may also include an enhancer sequence which is a DNA sequence capable of increasing the transcription of a gene into mRNA. The constructs of the present invention may contain a promoter alone or in combination with an enhancer, and these elements need not be contiguous.

A coding sequence is “under the control of” or is “operatively associated with” transcriptional and translational control sequences in a cell when RNA polymerase transcribes the coding sequence into RNA, which is then trans-RNA spliced (if it contains introns) and, if the sequence encodes a protein, is translated into that protein.

The term “express” and “expression” means allowing or causing the information in a gene or DNA sequence to become manifest, for example producing RNA (such as rRNA or mRNA) or a protein by activating the cellular functions involved in transcription and translation of a corresponding gene or DNA sequence. A DNA sequence is expressed by a cell to form an “expression product” such as an RNA (a mRNA or a rRNA) or a protein. The expression product itself, such as the resulting RNA or protein, may also said to be “expressed” by the cell.

The term “transfection” means the introduction of a foreign nucleic acid into a eukaryotic host cell. The term “transformation” means the introduction of a “foreign” (i.e., extrinsic or extracellular) gene, DNA or RNA sequence into a prokaryotic host cell so that the host cell will express the introduced gene or sequence to produce a desired substance, in this invention typically an RNA coded by the introduced gene or sequence, but also a protein or an enzyme coded by the introduced gene or sequence. The introduced gene or sequence may also be called a “cloned” or “foreign” gene or sequence, may include regulatory or control sequences such as, start, stop, promoter, signal, secretion or other sequences used by a cell's genetic machinery. The gene or sequence may include nonfunctional sequences or sequences with no known function. A host cell that receives and expresses introduced DNA or RNA has been “transformed” and is a “transformant” or a “clone”. The DNA or RNA introduced to a host cell can come from any source, including cells of the same genus or species as the host cell or cells of a different genus or species.

The terms “vector”, “cloning vector” and “expression vector” mean the vehicle by which a DNA or RNA sequence of a foreign gene can be introduced into a host cell so as to transform the host and promote expression of the introduced sequence. Vectors may include for example, plasmids, phages, and viruses and are discussed in greater detail below.

The term “expression system” means a host cell and compatible vector under suitable conditions, capable of expressing a protein coded for by foreign DNA carried by the vector and introduced to the host cell. Common expression systems include E. coli host cells and plasmid vectors, insect host cells such as Sf9, Hi5 or S2 cells and Baculovirus vectors, Drosophila cells (Schneider cells) and expression systems, and mammalian host cells and vectors.

The term “heterologous” refers to a combination of elements not naturally occurring. For example, the present invention includes chimeric RNA molecules that comprise an rRNA sequence and a heterologous RNA sequence which is not part of the rRNA sequence. In this context, the heterologous RNA sequence refers to an RNA sequence that is not naturally located within the ribosomal RNA sequence. Alternatively, the heterologous RNA sequence may be naturally located within the ribosomal RNA sequence, but is found at a location in the rRNA sequence where it does not naturally occur. As another example, heterologous DNA refers to DNA that is not naturally located in the cell, or in a chromosomal site of the cell. Preferably, heterologous DNA includes a gene foreign to the cell. A heterologous expression regulatory element is a regulatory element operatively associated with a different gene that the one it is operatively associated with in nature.

The term “homologous” refers to the relationship between two proteins that possess a “common evolutionary origin”, including proteins from superfamilies, such as the immunoglobulin superfamily, in the same species of organism, as well as homologous proteins from different species of organism (for example, myosin light chain polypeptide; see, Reeck et al., Cell, 50:667, 1987). Such proteins (and their encoding nucleic acids) have sequence homology, as reflected by their sequence similarity, whether in terms of percent identity or by the presence of specific residues or motifs and conserved positions.

The term “sequence similarity”, in all its grammatical forms, refers to the degree of identity or correspondence between nucleic acid or amino acid sequences that may or may not share a common evolutionary origin (see, Reeck et al., supra). However, in common usage and in the instant application, the term “homologous”, when modified with an adverb such as “highly”, may refer to sequence similarity and may or may not relate to a common evolutionary origin.

In specific embodiments, two nucleic acid sequences are “substantially homologous” or “substantially similar” when at least about 80%, and more preferably at least about 90% or at least about 95% of the nucleotides match over a defined length of the nucleic acid sequences, as determined by a sequence comparison algorithm known such as BLAST, FASTA, DNA Strider, CLUSTAL, etc. An example of such a sequence is an allelic or species variant of the specific genes of the present invention. Sequences that are substantially homologous may also be identified by hybridization, such as in a Southern hybridization experiment under stringent conditions as defined for that particular system.

Similarly, in particular embodiments of the invention, two amino acid sequences are “substantially homologous” or “substantially similar” when greater than 80% of the amino acid residues are identical, or when greater than about 90% of the amino acid residues are similar. Preferably the similar or homologous polypeptide sequences are identified by alignment using, for example, the GCG (Genetics Computer Group, Program Manual for the GCG Package, Version 7, Madison Wis.) pileup program, or using any of the programs and algorithms described above (for example, BLAST, FASTA, and CLUSTAL).

The terms “mutant” and “mutation” mean any detectable change in genetic material, such as DNA, or any process, mechanism or result of such a change. This includes gene mutations, in which the structure of a gene is altered, any gene or DNA arising from any mutation process, and any expression product, such as RNA, protein or enzyme, expressed by a modified gene or DNA sequence. The term “variant” may also be used to indicate a modified or altered gene, DNA sequence, RNA, enzyme, cell, or any kind of mutant. For example, the present invention relates to altered or “chimeric” RNA molecules that comprise an rRNA sequence that is altered by inserting a heterologous RNA sequence that is not naturally part of that sequence or is not naturally located at the position of that rRNA sequence.

The term “chimeric” is used herein in its usual sense: a construct or protein resulting from the combination of or fusion of genes from two or more different sources, in which the different parts of the chimera function together. The genes are fused, where necessary in-frame, in a single genetic construct. The present invention can be employed using any chimera of sHSPs, as long as the chimeric polypeptide retains the desired biological activity of chaperonin competency. The chimeric sHSPs of the present invention are comprised of fusions, for example, of fragments of different sHSPs from the same organism. A non-limiting example of such a sHSP chimera is an α-crystallin polypeptide in which its N-terminus has been replaced by the N-terminus of hsp 16.5. Chaperonin-competency can be determined by, for example, the ability of the chimeric sHSPs to increase the folding, secretion and/or expression of the protein to which they are fused. Methods for observing whether a protein a protein or polypeptide is expressed or secreted are readily available to the skilled artisan and examples of such methods are described herein.

Such chimeric sequences, as well as DNA and genes that encode them, are also referred to herein as “mutant” sequences.

“Sequence-conservative variants” of a polynucleotide sequence are those in which a change of one or more nucleotides in a given codon position results in no alteration in the amino acid encoded at that position.

“Function-conservative variants” of a polypeptide or polynucleotide are those in which a given amino acid residue in the polypeptide, or the amino acid residue encoded by a codon of the polynucleotide, has been changed or altered without altering the overall conformation and function of the polypeptide. For example, function-conservative variants may include, but are not limited to, replacement of an amino acid with one having similar properties (for example, polarity, hydrogen bonding potential, acidic, basic, hydrophobic, aromatic and the like). Amino acid residues with similar properties are well known in the art. For example, the amino acid residues arginine, histidine and lysine are hydrophilic, basic amino acid residues and may therefore be interchangeable. Similar, the amino acid residue isoleucine, which is a hydrophobic amino acid residue, may be replaced with leucine, methionine or valine. Such changes are expected to have little or no effect on the apparent molecular weight or isoelectric point of the polypeptide. Amino acid residues other than those indicated as conserved may also differ in a protein or enzyme so that the percent protein or amino acid sequence similarity between any two proteins of similar function may vary and may be, for example, from 70% to 99% as determined according to an alignment scheme such as the Cluster Method, wherein similarity is based on the MEGALIGN algorithm. “Function-conservative variants” of a given polypeptide also include polypeptides that have at least 60% amino acid sequence identity to the given polypeptide as determined sequence alignment algorithms such as the BLAST or FASTA algorithms.

Preferably, function-conservative variants of a given polypeptide have at least 75%, more preferably at least 85% and still more preferably at least 90% amino acid sequence identity to the given polypeptide and, preferably, also have the same or substantially similar properties, such as molecular weight and/or isoelectric point or functions, such as biological functions or activities, as the native or parent polypeptide to which it is compared.

As used herein, the term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled with radioactive nucleotides such as ³²P-nucleotides or nucleotides to which a label, such as biotin or a fluorescent dye (for example, Cy3 or Cy5) has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of a sHSP or to detect the presence of nucleic acids encoding sHSPs. Generally, oligonucleotides are prepared synthetically, preferably on a nucleic acid synthesizer. Accordingly, oligonucleotides can be prepared with non-naturally occurring phosphoester analog bonds, such as thioester bonds, etc.

A sequence that is “complementary” to a portion of a nucleic acid refers to a sequence having sufficient complementarity to be able to hybridize with the nucleic acid and form a stable duplex. The ability of nucleic acids to hybridize will depend both on the degree of sequence complementarity and the length of the antisense nucleic acid. Generally, however, the longer the hybridizing nucleic acid, the more base mismatches it may contain and still form a stable duplex (or triplex in triple helix methods). A tolerable degree of mismatch can be readily ascertained by using standard procedures to determine the melting temperature of a hybridized complex.

Specific non-limiting examples of synthetic oligonucleotides envisioned for this invention include, in addition to the nucleic acid moieties described above, oligonucleotides that contain phosphorothioates, phosphotriesters, methyl phosphonates, short chain alkyl, or cycloalkyl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—PO₂—O—CH₂). U.S. Pat. No. 5,677,437 describes heteroaromatic olignucleoside linkages. Nitrogen linkers or groups containing nitrogen can also be used to prepare oligonucleotide mimics (U.S. Pat. Nos. 5,792,844 and 5,783,682). U.S. Pat. No. 5,637,684 describes phosphoramidate and phosphorothioamidate oligomeric compounds. Also envisioned are oligonucleotides having morpholino backbone structures (U.S. Pat. No. 5,034,506). In other embodiments, such as the peptide-nucleic acid (PNA) backbone, the phosphodiester backbone of the oligonucleotide may be replaced with a polyamide backbone, the bases being bound directly or indirectly to the aza nitrogen atoms of the polyamide backbone (Nielsen et al., Science 254:1497). Other synthetic oligonucleotides may contain substituted sugar moieties comprising one of the following at the 2′ position: OH, SH, SCH₃, F, OCN, O(CH₂)ₙNH₂ or O(CH₂)ₙCH₃ where n is from 1 to about 10; C₁ to C₁₀ lower alkyl, substituted lower alkyl, alkaryl or aralkyl; Cl; Br; CN; CF₃; OCF₃; O-; S-, or N-alkyl; O-, S-, or N-alkenyl; SOCH₃; SO₂CH₃; ONO₂;NO₂; N₃; NH₂; heterocycloalkyl; heterocycloalkaryl; aminoalkylamino; polyalkylamino; substituted silyl; a fluorescein moiety; an RNA cleaving group; a reporter group; an intercalator; a group for improving the pharmacokinetic properties of an oligonucleotide; or a group for improving the pharmacodynamic properties of an oligonucleotide, and other substituents having similar properties. Oligonucleotides may also have sugar mimetics such as cyclobutyls or other carbocyclics in place of the pentofuranosyl group. Nucleotide units having nucleosides other than adenosine, cytidine, guanosine, thymidine and uridine, such as inosine, may be used in an oligonucleotide molecule.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ (melting temperature) of 55° C., can be used, along with 5×SSC, 0.1% SDS, 0.25% milk, and no formamide; or 30% formamide, 5×SSC, 0.5% SDS. Moderate stringency hybridization conditions correspond to a higher Tₘ, 40% formamide, with 5× or 6×SCC. High stringency hybridization conditions correspond to the highest Tₘ, 50% formamide, 5× or 6×SCC. SCC is a 0.15M NaCl, 0.015M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, such as oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

Suitable hybridization conditions for oligonucleotides, such as oligonucleotide probes or primers) are typically somewhat different than for full-length nucleic acids such as full-length cDNA, because of the oligonucleotides' lower melting temperature. Because the melting temperature of oligonucleotides will depend on the length of the oligonucleotide sequences involved, suitable hybridization temperatures will vary depending upon the oligoncucleotide molecules used. Exemplary temperatures maybe 37° C. (for 14-base oligonucleotides), 48° C. (for 17-base oligoncucleotides), 55° C. (for 20-base oligonucleotides) and 60° C. (for 23-base oligonucleotides). Exemplary suitable hybridization conditions for oligonucleotides include washing in 6×SSC/0.05% sodium pyrophosphate, or other conditions that afford equivalent levels of hybridization.

In a specific embodiment the “enhanced” expression or secretion of a folded, functional product is the increase in expression or secretion in the presence of sHSPs versus that in the absence of sHSPs.

### Polypeptides, Nucleic Acids, and Expression Vectors of the Present Invention

The present invention provides novel polypeptides, nucleic acids, and expression systems to enhance the expression and/or secretion of proteins or polypeptides in a host. In a preferred embodiment, the present invention relates to sHSP polypeptides that facilitate protein expression and secretion. In a further preferred embodiment, the sHSP polypeptide is a truncated α-crystallin polypeptide. This invention has been elucidated by the unexpected discovery of the unusual tertiary structure of α-crystallin and the unique ability of the N-terminal extension to control aggregation.

Therefore, the present invention relates to a method for increasing the expression and/or secretion of a protein or polypeptide present in a host cell, which includes expressing in the host cell a sHSP polypeptide and thereby increasing secretion of the protein or polypeptide.

The present invention also contemplates a method of increasing expression and/or secretion of a protein or polypeptide from a host cell by expressing a sHSP polypeptide encoded by an expression vector present in or provided to the host cell, thereby increasing the secretion of the protein or polypeptide.

The present invention further provides a method for increasing expression and/or secretion of protein or polypeptides from a host cell, which comprises expressing at least one sHSP polypeptide in the host cell. In one embodiment, the method of the invention comprises effecting the expression of at least one sHSP protein or polypeptide in a host cell, and cultivating the host cell under conditions suitable for expression and/or secretion of the protein or polypeptide. The expression of the sHSP polypeptide and the protein or polypeptide can be effected by inducing expression of a nucleic acid encoding the sHSP polypeptide and a nucleic acid encoding the protein or polypeptide wherein the nucleic acids are present in a host cell.

In another embodiment, the expression of the sHSP polypeptide and the protein or polypeptide are effected by introducing a first nucleic acid encoding the sHSP polypeptide and a second nucleic acid encoding a protein or polypeptide to be expressed into a host cell under conditions suitable for expression of the first and second nucleic acids. In a preferred embodiment, one or both of the first and second nucleic acids are present in expression vectors. In a further preferred embodiment, both the first and second nucleic acids are present in a single expression vector.

Small HSPs of the present invention include any sHSP that can facilitate or increase the expression and/or secretion of proteins. In particular, α-crystallin and thermophilic sHSPs are particularly preferred, as well as fragments thereof and chimeric proteins containing one or more of these polypeptides, proteins, or fragments. In a preferred embodiment, the sHSP is selected from wild-type α-crystallin, a truncated form of α-crystallin, a thermophilic sHSP, or a chimeric polypeptide containing one or more of these component polypeptides. In a most preferred embodiment, the sHSP is a truncated α-crystallin polypeptide lacking an N-terminal sequence present in the corresponding wild-type protein. In a further preferred embodiment, the truncated polypeptide of the invention has a sequence set forth in SEQ ID NO: 3, and the nucleic acid has a sequence set forth in SEQ ID NO: 2. Preferably, the truncated polypeptides is at least 117 amino acids in length, and more preferably, at least 121 amino acids. With respect to the N-terminal sequence, preferably residues of the wild-type N-terminal sequence have been deleted in the truncated polypeptide, and most preferably 51 residues. In an additional embodiment, the truncated wild-type N-terminal sequence may be between 1 and 56 residues.

Also contemplated are proteins, polypeptides, fragments or chimeras thereof that are substantially homologous to α-crystallin and thermophilic sHSPs and which are capable of enhancing or facilitating the expression and/or secretion of proteins or polypeptides in vitro. Procedures for observing whether a protein or polypeptide is expressed or secreted are readily available to the skilled artisan. For example, Goeddel, D. V. (Ed.) 1990, Gene Expression Technology, Methods in Enzymology, Vol 185, Academic Press, and Sambrook et al. 1989, Molecular Cloning: A Laboratory Manual, Vols. 1-3, Cold Spring Harbor Press, N.Y., provide procedures for detecting secreted protein or polypeptides. For example, to secrete a protein or polypeptide the host cell is cultivated under conditions sufficient for secretion of the protein or polypeptide. Such conditions include temperature, nutrient and cell density conditions that permit secretion by the cell. Moreover, such conditions are those under which the cell can perform basic cellular functions of transcription, translation and passage of proteins from one cellular compartment to another and are known to the skilled artisan.

Moreover, the skilled artisan will appreciate that an expressed or secreted protein or polypeptide can be detected in the culture medium used to maintain or grow the present host cells. The culture medium can be separated from the host cells by known procedures, such as centrifugation or filtration. The protein or polypeptide can then be detected in the cell-free culture medium by taking advantage of known properties characteristic of the protein or polypeptide. Such properties can include the distinct immunological, enzymatic or physical properties of the protein or polypeptide. For example, if a protein or polypeptide has a unique enzyme activity an assay for that activity can be performed on the culture medium used by the host cells. Moreover, when antibodies reactive against a given protein or polypeptide are available, such antibodies can be used to detect the protein or polypeptide in any known immunological assay (for example as in Harlowe, et al., 1988, Antibodies: A Laboratory Manual, Cold Spring Harbor Laboratory Press).

The expressed or secreted protein or polypeptide can also be detected using tests that distinguish proteins on the basis of characteristic physical properties such as molecular weight. To detect the physical properties of the protein or polypeptide all proteins newly synthesized by the host cell can be labeled, such as with a radioisotope. Common radioisotopes which are used to label proteins synthesized within a host cell include tritium, carbon-14, sulfur-35, and the like. For example, the host cell can be grown in ³⁵S-methionine or ³⁵S-cysteine medium, and a significant amount of the ³⁵S label will be preferentially incorporated into any newly synthesized protein, including the protein of interest. The ³⁵S-containing culture medium is then removed and the cells are washed and placed in fresh non-radioactive culture medium. After the cells are maintained in the fresh medium for a time and under conditions sufficient to allow secretion of the 31S— radiolabeled protein, the culture medium is collected and separated from the host cells. The molecular weight of the secreted labeled protein in the culture medium can then be determined by known procedures, such as polyacrylamide gel electrophoresis. Such procedures are described in more detail within Sambrook et al. (supra).

Thus, one of ordinary skill in the art can readily ascertain which sHSP polypeptides have sufficient homology to α-crystallin, thermophilic sHSPs, fragments thereof, or chimera comprising one or more of these polypeptides or fragments, to stimulate expression and/or secretion of a protein or polypeptide.

Purification of sHSP from natural or recombinant sources is achieved by methods well-known in the art, including, but not limited to, ion-exchange chromatography, reverse-phase chromatography on C4 columns, gel filtration, isoelectric focusing, affinity chromatography, and the like. sHSPs isolated from any source may be modified by methods known in the art. For example, sHSPs are phosphorylated or dephosphorylated, glycosylated or deglycosylated, and the like. Especially useful are modifications that alter solubility, stability, and binding specificity and affinity.

In an alternative embodiment of the present invention, the polypeptide described above optionally includes a linker sequence at the N-terminus which is designed to enhance the solubility of the polypeptide. The linker may be between 2 and 10 amino acid residues in length and preferably contains amino acids such as serine or glycine which are hydrophobic in nature in order to promote solubility of the sHSP in an aqueous environment.

Also provided is an isolated nucleic acid encoding a sHSP such as the truncated α-crystallin polypeptide described above, as well as an isolated nucleic acid that hybridizes, under stringent conditions, to the complement of a nucleic acid encoding the a sHSP, as set forth in SEQ ID NO: 2 (FIG. 2). In this regard, the invention further provides an oligonucleotide of at least 10 nucleotides which has a sequence complementary to a sequence present in the nucleic acid encoding a sHSP. Preferably, the oligonucleotide is at least 100 nucleotides in length, and more preferably, at least 200 or 300 nucleotides in length. In an alternate embodiment of the present invention, the oligonucleotide is detectably labeled. The detectable label may comprise any moiety capable of providing a signal, such as a visible signal, that the oligonucleotide is present. For example, the detectable label may be a radioisotope, a fluorophore, biotin, a chemiluminescent, or electrochemiluminescent label.

Examples of protein or polypeptides which are preferably expressed and/or secreted by the present methods include mammalian protein or polypeptides such as enzymes, cytokines, growth factors, hormones, vaccines, antibodies and the like. More particularly, preferred overexpressed protein or polypeptides of the present invention include protein or polypeptides such as erythropoietin, insulin, somatotropin, growth hormone releasing factor, platelet derived growth factor, epidermal growth factor, transforming growth factor, alpha, transforming growth factor, beta., epidermal growth factor, fibroblast growth factor, nerve growth factor, insulin-like growth factor I, insulin-like growth factor II, clotting Factor VIII, superoxide dismutase, alpha-interferon, gamma-interferon, interleukin-1, interleukin-2, interleukin-3, interleukin-4, interleukin-5, interleukin-6, granulocyte colony stimulating factor, multi-lineage colony stimulating activity, granulocyte-macrophage stimulating factor, macrophage colony stimulating factor, T cell growth factor, lymphotoxin and the like. For medical applications, preferred protein or polypeptides are human protein or polypeptides, however other protein or polypeptides may be used for industrial applications.

The present invention also provides vectors that include nucleic acids encoding sHSPs of the invention in part or in whole. The vector may include a nucleic acid encoding a sHSP, a thermophilic sHSP, HSP16.5, α-crystallin, truncated α-crystallin, or chimera containing one or more of the same, and optionally, a nucleic acid encoding a protein of interest. Such vectors include, for example, plasmid vectors for expression in a variety of eukaryotic and prokaryotic hosts. The vector also further comprises an expression control sequence operably linked to the nucleic acid. The vectors of the present invention may be incorporated into a host cell, which is either a eukaryotic or a prokaryotic cell. Preferably, the host cell is either E. coli, yeast, COS cells, PC12 cells, CHO cells, or GH4C1 cells.

Another embodiment of the invention provides a plasmid vector having a nucleic acid encoding a sHSP and a nucleic acid encoding a protein or polypeptide operatively associated with an expression control sequence.

Suitable vectors for use in practicing the present invention include, without limitation, YEp352, pcDNAI (Invitrogen, Carlsbad, Calif. CA1, pRc/CMV (Invitrogen), and pSFV1 (GIBCO/BRL, Gaithersburg, Md.). One preferred vector for use in the invention is pSFV1. Suitable host cells include E. coli, yeast, COS cells, PC12 cells, CHO cells, GH4C1 cells, EHK-21 cells, and amphibian melanophore cells. BHK-21 cells are a preferred host cell line for use in practicing the present invention. Suitable vectors for the construction of naked DNA or genetic vaccinations include without limitation pTarget (Promega, Madison, Wis.), pSI (Promega, Madison, Wis.) and pcDNA (Invitrogen, Carlsbad, Calif.).

Nucleic acids encoding the sHSP(s) polypeptide(s) of the invention, alone or in combination with a protein of interest, may also be introduced into cells by recombination events. For example, such a sequence is microinjected into a cell, effecting homologous recombination at the site of an endogenous gene encoding the polypeptide, an analog or pseudogene thereof, or a sequence with substantial identify to an sHSP-encoding gene. Other recombination-based methods such as non-homologous recombinations, and deletion of endogenous gene by homologous recombination, especially in pluripotent cells, are also used.

Additionally, an sHSP-encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Modifications can also be made to introduce restriction sites and facilitate cloning the sHSP gene into an expression vector. Any technique for mutagenesis known in the art can be used, including but not limited to, in vitro site-directed mutagenesis (Hutchinson, C., et al., J. Biol. Chem. 253:6551, 1978; Zoller and Smith, DNA 3:479-488, 1984; Oliphant et al., Gene 44:177, 1986; Hutchinson et al., Proc. Natl. Acad. Sci. U.S.A. 83:710, 1986), use of TAB″ linkers (Pharmacia), etc. PCR techniques are preferred for site directed mutagenesis (see Higuchi, (1989), “Using PCR to Engineer DNA”, in PCR Technology: Principles and Applications for DNA Amplification, H. Erlich, ed., Stockton Press, Chapter 6, pp. 61-70).

The identified and isolated gene can then be inserted into an appropriate cloning vector. A large number of vector-host systems known in the art may be used. Possible vectors include, but are not limited to, plasmids or modified viruses, but the vector system must be compatible with the host cell used. Examples of vectors include, but are not limited to, E. coli, bacteriophages such as lambda derivatives, or plasmids such as pBR322 derivatives or pUC plasmid derivatives, such as pGEX vectors, pmal-c, pFLAG, pKK plasmids (Clonetech), pET plasmids (Novagen, Inc., Madison, Wis.), pRSET or pREP plasmids, pcDNA (Invitrogen, Carlsbad, Calif.), or pMAL plasmids (New England Biolabs, Beverly, Mass.), etc. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences.

Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, such as E. coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences form the yeast 2 m plasmid.

A nucleotide sequence coding for a sHSP, alone or in combination with a protein of interest may be inserted into an appropriate expression vector, such as a vector which contains the necessary elements for the transcription and translation of the inserted protein-coding sequence. Thus, a nucleic acid encoding an sHSP of the invention can be operationally associated with a promoter in an expression vector of the invention. Both cDNA and genomic sequences can be cloned and expressed under control of such regulatory sequences. Such vectors can be used to express functional or functionally inactivated sHSPs. The necessary transcriptional and translational signals can be provided on a recombinant expression vector.

Potential host-vector systems include, but are not limited to, mammalian or other vertebrate cell systems transfected with expression plasmids or infected with virus (such as vaccinia virus, adenovirus, adeno-associated virus, herpes virus, etc.); insect cell systems infected with virus (such as baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

Expression of an sHSP may be controlled by any promoter/enhancer element known in the art, but these regulatory elements must be functional in the host selected for expression. Promoters which may be used to control sHSP gene expression include, but are not limited to, cytomegalovirus (CMV) promoter (U.S. Pat. Nos. 5,385,839 and 5,168,062), the SV40 early promoter region (Benoist and Chambon, Nature, 290:304-310, 1980), the promoter contained in the 3′ long terminal repeat of Rous sarcoma virus (Yamamoto, et al., Cell 22:787-797, 1980), the herpes thymidine kinase promoter (Wagner et al., Proc. Natl. Acad. Sci. U.S.A. 1981, 78:1441-1445, 1981), the regulatory sequences of the metallothionine gene (Brinster et al., Nature, 296:39-42, 1982); prokaryotic expression vectors such as the β-lactamase promoter (Villa-Komaroff, et al., Proc. Natl. Acad. Sci. U.S.A., 75:3727-3731, 1978), or the tac promoter (DeBoer, et al., Proc. Natl. Acad. Sci. USA., 80:21-25, 1983); see also “Useful proteins from recombinant bacteria” in Scientific American, 242:74-94, 1980. Still other useful promoter elements which may be used include promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit hematopoietic tissue specificity, in particular: beta-globin gene control region which is active in myeloid cells (Mogram et al., Nature, 315:338-340, 1985; Kollias et al., Cell, 46:89-94, 1986), hematopoietic stem cell differentiation factor promoters, erythropoietin receptor promoter (Maouche et al., Blood, 15:2557, 1991).

Indeed, any type of plasmid, cosmid, YAC or viral vector may be used to prepare a recombinant nucleic acid construct which can be introduced to a cell, or to tissue, where expression of an sHSP protein or polypeptide is desired. Alternatively, wherein expression of a recombinant sHSP protein or polypeptide in a particular type of cell or tissue is desired, viral vectors that selectively infect the desired cell type or tissue type can be used.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, such as E. coli plasmids col E1, pCR1, pBR322, pMal-C2, pET, pGEX (Smith et al., Gene, 67:31-40, 1988), pCR2.1 and pcDNA 3.1+(Invitrogen, Carlsbad, Calif.), pMB9 and their derivatives, plasmids such as RP4; phage DNAs, such as the numerous derivatives of phage 1, for example NM989, and other phage DNA, such as M13 and filamentous single stranded phage DNA; yeast plasmids such as the 2 m plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Preferred vectors are viral vectors, such as lentiviruses, retroviruses, herpes viruses, adenoviruses, adeno-associated viruses, vaccinia virus, baculovirus, and other recombinant viruses with desirable cellular tropism. Thus, a gene encoding a functional or mutant sHSP can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in International Patent Publication WO 95/28494, published October 1995.

Viral vectors commonly used for in vivo or ex vivo targeting and therapy procedures are DNA-based vectors and retroviral vectors. Methods for constructing and using viral vectors are known in the art (see, Miller and Rosman, BioTechniques, 7:980-990, 1992). Preferably, the viral vectors are replication defective, that is, they are unable to replicate autonomously in the target cell. In general, the genome of the replication defective viral vectors which are used within the scope of the present invention lack at least one region which is necessary for the replication of the virus in the infected cell. These regions can either be eliminated (in whole or in part), be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome which are necessary for encapsidating the viral particles.

DNA viral vectors include an attenuated or defective DNA virus, such as, but not limited to, herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al., Molec. Cell. Neurosci., 2:320-330, 1991), defective herpes virus vector lacking a glyco-protein L gene (Patent Publication RD 371005 A), or other defective herpes virus vectors (International Patent Publication No. WO 94/21807, published Sep. 29, 1994; International Patent Publication No. WO 92/05263, published Apr. 2, 1994); an attenuated adenovirus vector, such as the vector described by Stratford-Perricaudet et al. (J. Clin. Invest. 90:626-630, 1992; see also La Salle et al., Science, 259:988-990, 1993); and a defective adeno-associated virus vector (Samulski et al., J. Virol., 61:3096-3101, 1987; Samulski et al., J. Virol. 63:3822-3828, 1989; Lebkowski et al., Mol. Cell. Biol., 8:3988-3996, 1988).

Various companies produce viral vectors commercially, including but by no means limited to Avigen, Inc. (Alameda, Calif.; AAV vectors), Cell Genesys (Foster City, Calif.; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), Transgene (Strasbourg, France; adenoviral, vaccinia, retroviral, and lentiviral vectors) and Invitrogen (Carlbad, Calif.).

In another embodiment, the vector can be introduced in vivo by lipofection, as naked DNA, or with other transfection facilitating agents (peptides, polymers, etc.). Synthetic cationic lipids can be used to prepare liposomes for in vivo transfection of a gene encoding a marker (Felgner et al., Proc. Natl. Acad. Sci. U.S.A., 84:7413-7417, 1987; Felgner and Ringold, Science, 337:387-388, 1989; Mackey et al., Proc. Natl. Acad. Sci. U.S.A., 85:8027-8031, 1988; Ulmer et al., Science, 259:1745-1748, 1993). Useful lipid compounds and compositions for transfer of nucleic acids are described in International Patent Publications WO 95/18863 and WO 96/17823, and in U.S. Pat. No. 5,459,127. Lipids may be chemically coupled to other molecules for the purpose of targeting (see, Mackey et al., Proc. Natl. Acad. Sci. U.S.A., 85:8027-8031, 1988). Targeted peptides, such as hormones or neurotransmitters, and proteins such as antibodies, or non-peptide molecules could be coupled to liposomes chemically. Other molecules are also useful for facilitating transfection of a nucleic acid in vivo, such as a cationic oligopeptide (see International Patent Publication WO 95/21931), peptides derived from DNA binding proteins (see International Patent Publication WO 96/25508), or a cationic polymer (see International Patent Publication WO 95/21931).

It is also possible to introduce the vector in vivo as a naked DNA plasmid. Naked DNA vectors for gene therapy can be introduced into the desired host cells by methods known in the art, such as electroporation, microinjection, cell fusion, DEAE dextran, calcium phosphate precipitation, use of a gene gun, or use of a DNA vector transporter (see, Wu et al., J. Biol. Chem., 267:963-967, 1992; Wu and Wu, J. Biol. Chem., 263:14621-14624, 1988; Hartmut et al., Canadian Patent Application No. 2,012,311, filed Mar. 15, 1990; Williams et al., Proc. Natl. Acad. Sci. U.S.A., 88:2726-2730, 1991). Receptor-mediated DNA delivery approaches can also be used (Curiel et al., Hum. Gene Ther., 3:147-154, 1992; Wu and Wu, J. Biol. Chem., 262:4429-4432, 1987). U.S. Pat. Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir et al., C.P. Acad. Sci., 321:893, 1998; WO 99/01157; WO 99/01158; WO 99/01175).

In a preferred embodiment, the method of the present invention are particularly well suited for use in E. coli. However, any host may be used to enhance expression and/or secretion of a protein or polypeptide. For example, bacteria other then E. Coli such as Bacillus subtillus, yeast, or insect cell lines such as SF-3 or SF-4.

### Applications and Uses

Described herein are various applications and uses for sHSPs, including applications and uses for sHSP nucleic acids, polypeptides, and expression systems. As described in the Examples, infra, the sHSPs of the present invention may enhance protein expression and/or secretion. In particular, the molecules of the invention may be used to enhance expression of otherwise unstable proteins, such as insulin, alcohol dehydrogenase, lactate dehydrogenase and carbonic anhydrase, which tend to aggregate upon expression. It is important to note that the foregoing list of proteins that may be used in the methods of the present invention is merely illustrative, and is not intended to limit the scope of the invention. It will be understood that by virtue of the way in which the molecules of the invention enhance protein expression, they may be used to enhance expression of virtually any protein, natural or synthetic, having a tendency to aggregate upon expression in a host.

With respect to enhancement of protein expression, the molecules of the present invention are capable of increasing expression of a wild-type protein by at least about 10%, preferably 25%, and more preferably several fold. In particular, the molecules of the invention enhance the amount of a protein that is expressed in a host cell that is soluble, i.e., non-aggregated. Preferably, the molecules may enhance solubility by at least 10%, preferably 50%, and most preferably several fold.

In addition, the molecules of the present invention may be used to create a thermophilic host which tolerates elevated temperatures. In this regard, the molecules of the invention will be expressed at elevated temperatures to stabilize and enhance expression of proteins in the thermophilic host. Preferably the molecules of the present invention enhance thermal stability of the host by at least five degrees Celsius and more preferably ten degrees Celsius.

### EXAMPLES

The present invention is also described by means of particular examples. However, the use of such examples anywhere in the specification is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification and can be made without departing from its spirit and scope. The invention is therefore to be limited only by the terms of the appended claims along with the full scope of equivalents to which the claims are entitled.

### Example 1

### Methods

Modeling. Initial sequence alignments were generated using the multiple alignment programs PILEUP and CLUSTAL W and the pairwise alignment program ALINORM, which makes use of sequence information and secondary structure prediction. Obvious errors caused by the presence of large insertions and strand confusion were repaid manually.

Structural modeling based on the resulting alignments and the thermophilic small heat shock protein structure determined by Kim et al. (Nature, 394(6693):595-599, 1998) was carried out using the InsightII/homology modeling package from Molecular Simulations. Alternative alignments were examined for their ability to produce reasonable structures by stearic and energetic criteria, to correctly orient residues based on their hydrophobicity, and to correctly position conserved residues involved in key structural interactions, such as ion pairs and H-bonds. Magnetic resonance information from spin label studies (Berengian, et al., J. Biol. Chem., 274(10):6305-6314, 1999) was used to select between similar alternative alignments, such as selection of β strand start positions.

Crude model structures were refined using the Discover module of Molecular Simulations InsightII/homology package. Refinement included splice point repairs to produce favorable bond genometrics, and energy minimization carried out on all atoms except the backbone atoms in regions of conserved secondary structure.

PCR amplification. Oligonucleotide sequences were designed to anneal specifically to the alpha A crystallin gene (bovine); such that, the 5′ oligonucleotide would begin amplification at residue 51, in order to eliminate the N-terminal region. The 3′ oligonucleotide incorporates the alpha A crystallin stop codon and introduces an XhoI site. After endonuclease digestion with XhoI, the length of the predicted alpha A crystallin protein or polypeptide is 124 residues. The oligonucleotide sequences used were the following:

| | |
|---|---|
| upstream | |
| 5′-TCCCTCTTCCGCACCGTGCTGG-3′ | (SEQ ID NO: 13) |
| downstream | |
| 5′-GCTTTGTTAGCAGCTCGAGCCTTAGGACGA | (SEQ ID NO: 14) |
| G-3′ | |

Additionally, a 15 residue linker region, containing a start codon and preceded by an NdeI site, was attached 5′ to the N-terminally deleted alpha A gene discussed above (using overlap extension amplification). The sequences of the serine/glycine linker oligonucleotides were the following:

| | |
|---|---|
| upstream | |
| 5′-CATATGGACGTCACCACCGGAACCGGAACC | (SEQ ID NO: 15) |
| ACCGGAACCACCGCTAGC-3′ | |
| downstream | |
| 5′-CCAGCACGGTGCGGAAGAGGGAGCTAGCGG | (SEQ ID NO: 16) |
| TGGTTCCGGT-3′ | |

The total length of the alpha A crystallin_{Δ51+} construct is 139 residues. The sequence of the alpha A crystallin_{Δ51+} gene was verified using an ABI 373 sequencer. The T7 promoter primer (upstream) and the T7 terminator primer (downstream), (see Novagen) anneal to the pet20b vector.

Protein expression andpurification. The alpha A crystallin_{Δ51+} gene was ligated into the pet20b vector (Novagen) and subsequently transformed into the E. coli expression strain BL21 (DE3) pLysS. Cell lysis and supernatant preparation was conducted according to Horowitz et al. (34). Protein supernatant was applied, at ˜2.0 ml/min, to a Hiprep 16/10 Q XL column (Amersham/Pharmacia) that had been equilibrated with 20 mM Tris-100 mM NaCl. The Δ51+ constructed protein eluted in 350 mM NaCl and these fractions were applied to ˜100 ml bed volume column packed with Sephacryl S-400 gel filtration material. The column was equilibrated with 20 mM Tris-250 mM NaCl, and elution carried out at ˜1.0 m/min.

Aggregate size. The size of the alpha A crystallin_{Δ51+} protein was determined using a Superose 12 HR 10/30 gel exclusion column (Amersham-Pharmacia biotech). To calibrate the Superose 12 HR 10/30 column the following protein standards were run through at 0.5 mlmin in 20 mM Tris, pH 8.0 and 200 mM NaCl: B-Amylase 200,000; Bovine Serum Albumin 66,000; Carbonic Anhydrase 29,000, and Cytochrome C 12,400. The purified alpha A crystallin_{Δ51+} protein construct was then run through the column using the same buffer, sample volume (150 ul) and flow rate.

Aggregation assays. The ability of the alpha A crystallin_{Δ51+} protein to prevent protein aggregation, as compared to wild type A crystallin, was assayed, in vitro, using a 4.5:1 alpha A crystallin_{Δ51+} protein (19.4 uM) to insulin (87.2) ratio. Proteins were dialyzed in 50 mM imidazole, 100 mM NaCl, 0.02% NaN₃, at pH 7.5. Reactions were initiated, on a 96-well flat bottom well plate, by the addition of 20 mM DTT, at 25° C., using a Spectra Max 190 plate reader. Absorbencies were read at 360 over a 60 minute time period.

### Results

Sequence Homology. FIG. 4 (Sutton, et al., Science, 273:1058-1073, 1996; Tseng, et al., Plant Mol. Bio., 18:963-965, 1992) shows a subset of an extensive multiple alignment produced by manual adjustment of the output of several programs (PILEUP, CLUSTAL W, AN ALINORM) (Koetz, et al., Invest. Opthalmol. Vis. Sci. (ARVO suppl), 39:S1018, 1998 and Salerno, et al., Protein Sci. 8 (suppl. 1): 125, 1999). Several features of this alignment are of particular importance in understanding structural similarities in the sHSP superfamily, most notably a common structural motif that extends further toward the N terminal than previously believed (Koetz, et al., Invest. Opthalmol. Vis. Sci. (ARVO suppl.), 39:S1018, 1998 and Salerno, et al., Protein Sci. 8 (suppl. 1):125, 1999). The region of homology in all proteins examined not only includes the region covered by the second and third exons in α-crystallin but also includes some similarities in the first exon, although no extensive homology is present near the N-terminus. Additionally, in the smallest members of the superfamily, a very short N terminal sequence, including fewer than ten residues, precedes the onset of homology with crystallins. Finally, it appears that in α-crystallins, fewer than forty residues precede the region of homology observed with other heat shock proteins.

The smallest members of the superfamily are single domain structures dominated by β sheet motifs, since they display homology to the core domain structure in HSP16.5. Since the α-crystallins are homologous to these small proteins for three-quarters of their length, it follows that the structures of the α-crystallins are similar to the smaller proteins, with some additional insertions and a significant N-terminal extension. Since the α-crystallin-terminal extension is at most forty residues in length, it appears that there is insufficient material in the N-terminal extension for an independently folded domain to be present. Thus, α-crystallin is a single domain structure with an N-terminal sequence motif. Regardless of the structure of the N-terminal extension of α-crystallin, it is unlikely to be stably folded in the absence of the remainder of the sequence.

Larger members of the sHSP superfamily have molecular weights of 25-27 kD. The two-fold difference in size between these and the smallest HSPs reflects N and C terminal extensions too small to be domains, combined additionally with internal insertions corresponding to extended loop regions between units of conserved secondary structure (FIG. 4). Since the homology to α-crystallin extends to within twenty residues of the N and C terminals in these proteins, there is not sufficient material at the N or C terminals to form independently folded second domains. Therefore, the members of the sHSP superfamily are single domain proteins; a few heat shock proteins, having molecular weights of approximately 40 kD, contain two homologous repeats.

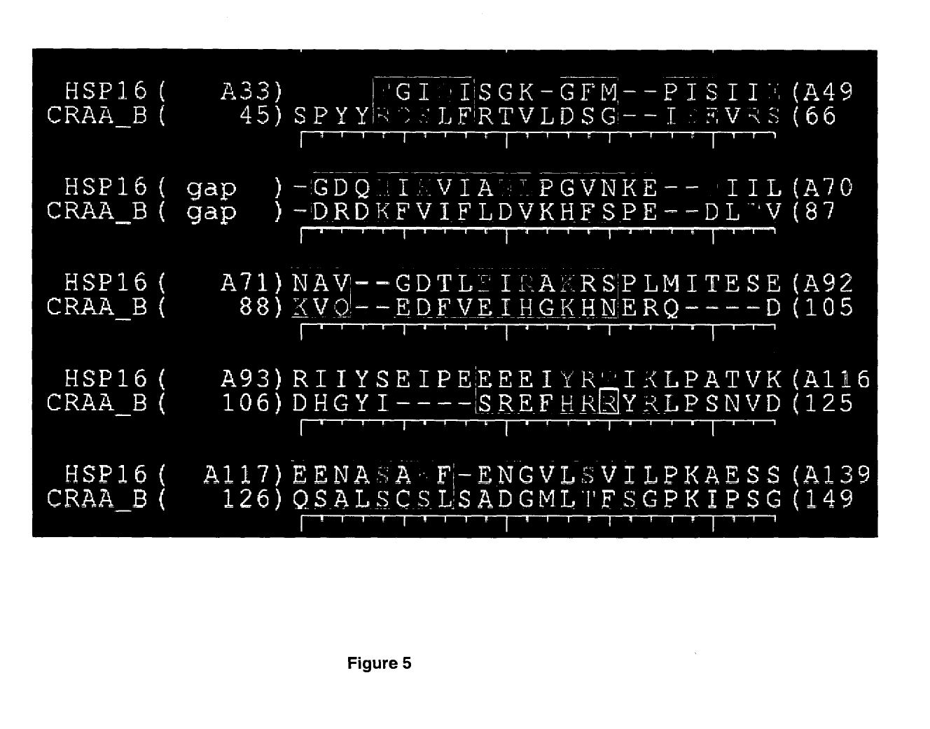

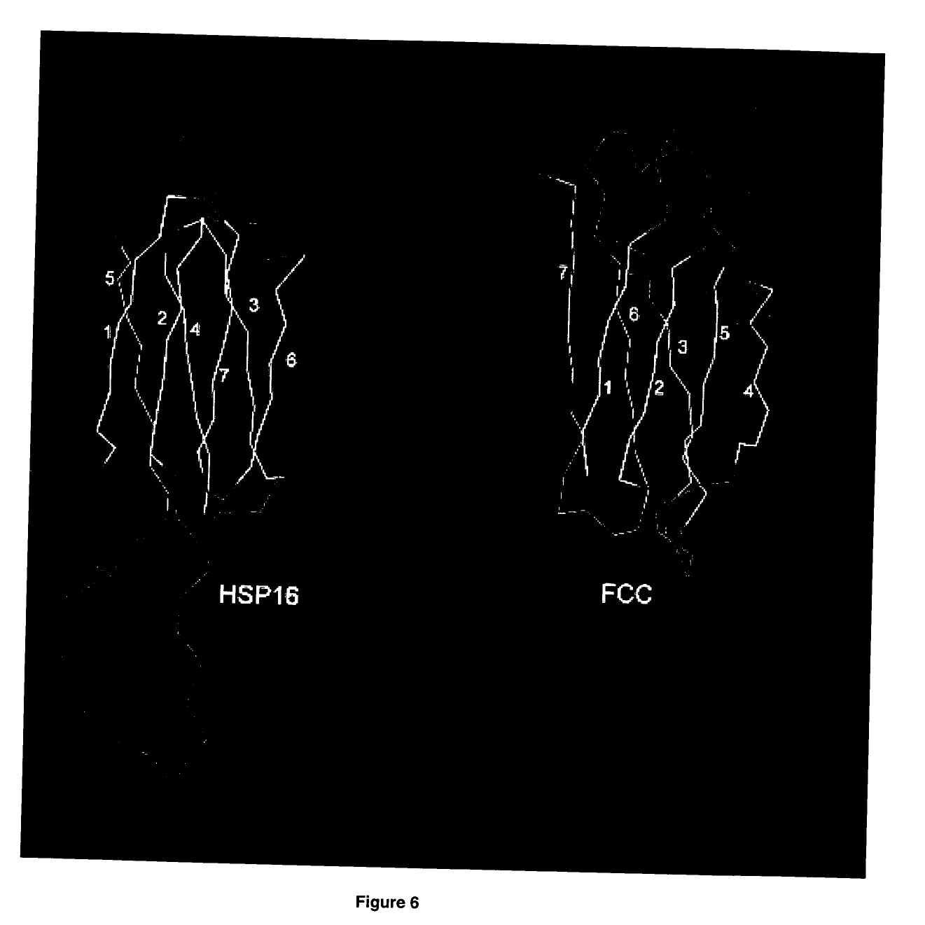

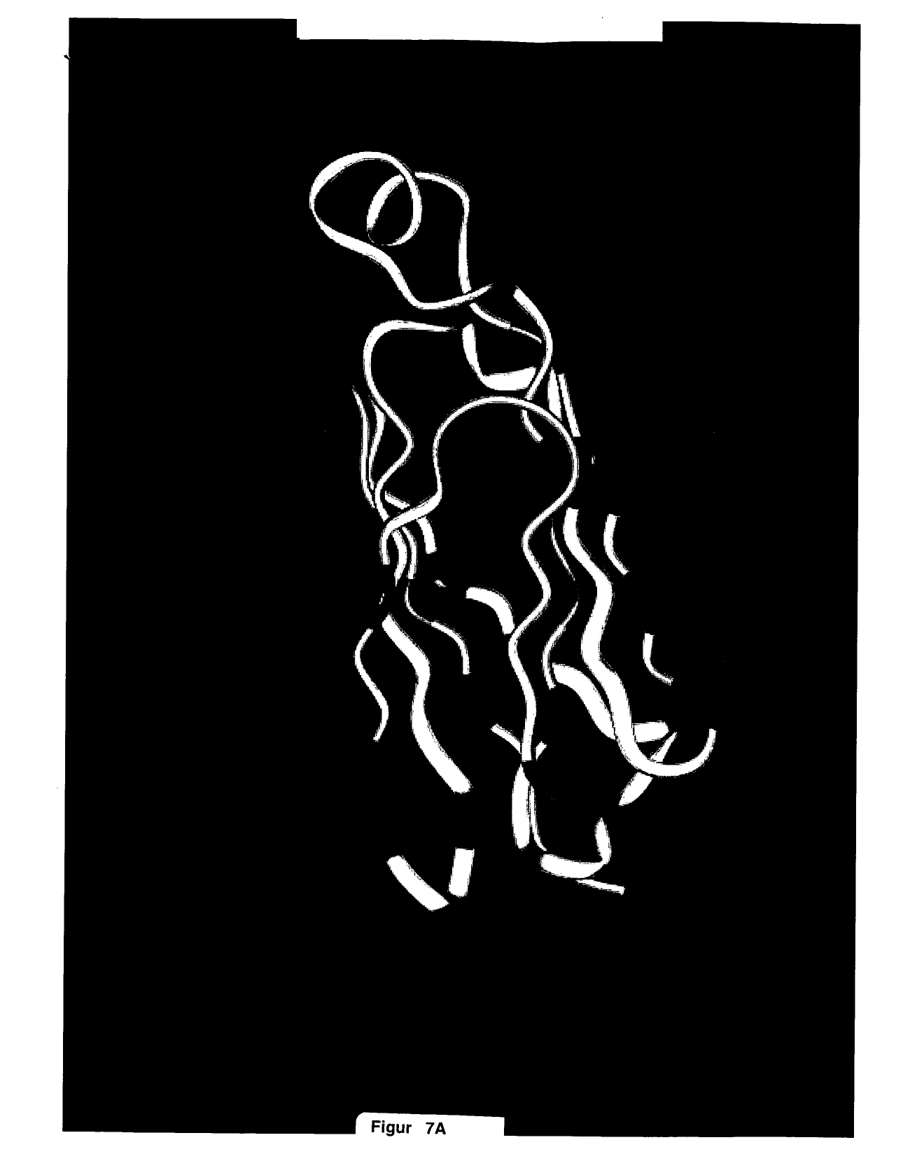

Homology modeling to the crystal structure of a sHSP. Kim et al. (Nature, 394(6693):595-599, 1998) used a sequence alignment obtained from PILEUP to assign secondary structure to eight other sequences based on their crystal structure. These included examples of αA and αB crystallin, the latter of which has been used in molecular modeling (Muchowki, et al., J. Mol. Bio., 289:397-411, 1999). Large scale multiple alignments (as shown in FIG. 4), however, suggest that their assignment of secondary structure to the α-crystallins and other sHSPs may contain errors which affect the first few β strands. FIG. 5 shows a subset of the sequences from FIG. 4 with an alternative assignment of the strands. Note in particular that the previous alignment places the sHSP and rodent inserts within β strands, which is generally not favored. Schematic topology maps for the Kim et al. structure and the α-crystallin structure are shown in FIG. 6 (left). It should be noted that while these structures superficially resemble the immunoglobulin fold (Moron, et al., Int. J. Biol. Macromol., 2(3-4):219-227, 1998), the folding topologies of the β sheets are actually quite different (FIG. 6, right). None of the sHSP superfamily members has an immunoglobulin fold.

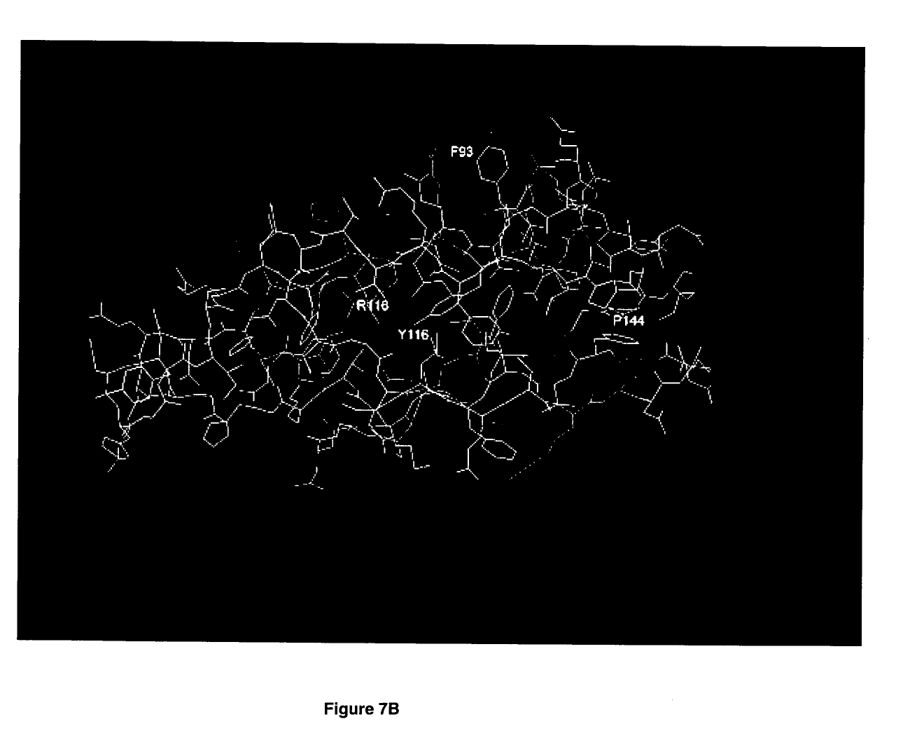

FIG. 7 shows a homology based model for αA crystallin based on the structure of Kim et al (Nature, 394(6693):595-599, 1998). The outstanding features of the sHSP 16.5 structure have been preserved while generating a sterically and energetically plausible model. Relaxation readily led to the removal of all ‘bmps’, and generated a free energy of approximately 300 kcal/mol using van Waals and electrostatic terms. The outstanding feature of the core structure is the two sheets, formed by alternating sequence elements, and enclosing an almost entirely hydrophobic core. The surface of this brick-like structure is largely hydrophilic, but contains hydrophobic patches, which almost certainly function in aggregation. The loop containing β6, the strand involved in dimerization in sHSP 16.5 is much shorter in α-crystallin (14 residues vs. 23 residues) and cannot possibly form the same dimer-promoting structure. It is still the longest loop between two strands, however, and is likely to play a role in formation of a dimer with altered properties, which may include different geometry, increased flexibility, and lower stability.

The model is capable of rationalizing prior mutant data on α-crystallin. Most crystallin mutants show little or no difference when compared to the native protein. The dominance of relatively non-specific hydrophobic interactions and the presence of numerous interactions promoting structural integrity tend to make the structure impervious to changes in side chain size with the same properties and resistant to most changes in side chain type because of extensive forms of stabilization. Comparison of the model structure with the HSP 16.5 structure reveals a small number of potential conserved hydrogen bonds, which may be critical for the preservation of the common core structure (see Table 1).

TABLE 1
Side-chain hydrogen bonds conserved in HSP 16.5
and αA-crystallin

| | |
|---|---|
| HSP 16.5 | α A-crystallin |

| | | | |
|---|---|---|---|
| N64 | E66 | S81 | E83 |
| N71 | E78 | K88 | E95 |
| R83 | F42 | H100 | K78 |
| R83 | D61 | H100 | S111 |
| R107 | G41 | R116 | D58 |
| R107 | M43 | R116 | G60 |
| K110 | D75 | R119 | D92 |
| T114 | S139 | N123 | S148 or G149 |

The only critical mutations that affect these structures are the R120G and R116G mutants (Bova, et al., Proc. Natl. Acad. Sci. U.S.A., 96(11):6137-6142, 1999), which greatly decrease the stability of the native structure. R116 is located in an interior strand, and is unusual in that it is a hydrophilic residue that is directed into the core. The function of R116 in HSP 16.5 is to form an hydrogen bond to the backbone of the loop between the first and second P strands, and in doing so it stabilizes the turn and anchors the sheets together.

These observations are consistent with the magnetic resonance data of Berengian et al. (Biol. Chem. 274(10):6305-6314, 1999), who used spin labeled α-crystallin to study the proximity of residues and deduce the positions of sheets in the structure. The region which forms the first two strands in the structure HSP 16.5 is difficult to align with a large number of sHSP sequences in a way which can be readily reconciled with these data. In order to reconcile the position of the strands corresponding to β2 and β3 of HSP 16.5, Berengian et al. (Biol. Chem. 274(10):6305-6314, 1999) were forced to choose an alignment which, extended to the rodent α-crystallins, forces a large insertion into a β sheet. If this is correct, rodent crystallins have a large β blowout on the edge of the β sheet in a position that Berengian et al. believe is important in interactions between subunits. This is unlikely.

Berengian et al. failed to detect the interactions that would be expected from residues forming a strand equivalent to strand 1 in HSP 16.5, and conclude that such a strand is not present in α-crystallin. This is possible; however, the conserved and critical residue equivalent to R116 functions in HSP 16.5 to stabilize the loop connecting β1 and it was found that the bulkier side chains of the crystallin made it difficult to construct such an element on an HSP 16.5 template. The model that resulted, shown as part of FIG. 7, has an extended loop, which incorporates the R116H bond and a short β strand cap with only three residues. The pattern of insertions in this region restricts the possibility of conserved β1-like structures to the sequence region chosen.

Also of interest is the highly conserved PK sequence which follows the core domain in sHSPs. This sequence is a strong helix initiator, which forms a cap at the N-terminal end of the short second helix of HSP 16.5. Its presence in α-crystallin suggests the possibility of a short helix. It is followed in HSP 16.5 by a terminal β strand that is no part of a sheet, but which mediates the formation of higher order aggregates by inserting two hydrophobic residues into the interior of a neighboring dimer. No comparable structure exists in the corresponding position of α-crystallin sequences, but about ten residues towards the C-terminal there is a conserved IPI sequence, which could perform the same function. If this sequence does interact with nearby dimers, the longer linker connecting it to the core would suggest a different aggregation geometry.

Role of the N-terminal in aggregation—design of a soluble α-crystallin construct. A major difference in the primary structure of α-crystallins and related small heat shock proteins which form small, well-ordered aggregates is the extent of the hydrophobic N-terminal tail which precedes the onset of the common domain. Calculations indicate that the N-terminal regions of α-crystallins are too large to pack inside the compact aggregates of other small heat shock proteins. This suggests that the N-terminal volume is a major controlling factor of aggregation in the sHSP superfamily.

To test the model described above and the observations derived from it, a crystallin variant was constructed to examine the role of a specific region in the sequence in folding and aggregation. Alignments suggest that the earliest residue likely to be involved in formation of the stably folded core domain is residue 52; accordingly, a truncated crystallin gene was constructed by per amplification in which the base pairs coding for the first 51 residues were replaced by a short sequence corresponding to a 15 residue serine-glycine tail to improve solubility.

Alpha A crystallin linker protein was expressed in soluble form in E. coli BL21 (DE3) pLysS transformed with Novegen pet20b vector containing the modified gene. Purification of the construct from lysed cells by ion exchange and gel exclusion chromatography steps was straightforward. Unlike all previous truncated α-crystallin constructs, the α-crystallin_{Δ51+} expressed at levels comparable to the holoprotein, and could be purified in high yield; in both cases, 20 mg of pure protein can be readily obtained from a one liter cell culture. SDS PAGE gels indicate that the α-crystallin is by far the most heavily expressed protein in the cell, and probably accounts for about half of the total cell protein. This level of expression of soluble protein strongly suggests stable folding of the core domain.

The aggregate size of the purified protein, determined by Superose 13HR chromatography, was calculated to be 60,000 daltons, which corresponds in size to a tetramer. The corresponding aggregate size of wild type α-crystallin is about 800,000 daltons, depending on solution conditions. This strongly supports the suggestion that the large N-terminal hydrophobic extension of α-crystallin is responsible for the formation of the large disordered aggregates seen with the wild type protein.

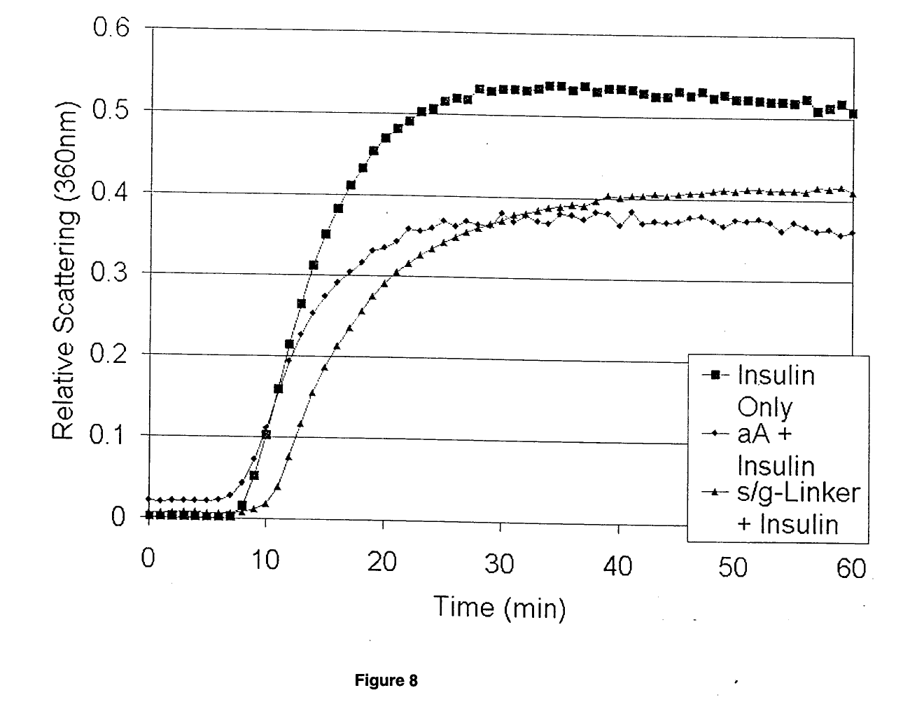

As shown in FIG. 8, the construct α-crystallin_{Δ51+} constructs indicates that the construct is at least as effective at reducing insulin aggregation as indicated by scattering at 360 nm. The N-terminal region is not essential for function as a heat shock protein. This is consistent with the homology-based observations comparing α-crystallin to the smallest members of the superfamily, which have short N-terminal tails comparable in size to the serine-glycine tail of the construct. It is also consistent with a picture in which the hydrophobic N-terminal tail is packed inside the disordered aggregate of the wild type protein, and suggests that externally located sequence regions are responsible for chaperonin-like activity.

Information from an α-crystallin/hsp 16.5 chimera (51, 51) is also relevant to understanding the role of the N-terminal region. Replacement of the N-terminal region of α-crystallin with the corresponding region of hsp 16.5 failed to produce small aggregates, but did produce a chaperonin-competent, highly expressed protein. Replacement of the N-terminus of hsp 16.5 with the corresponding region of α-crystallin produced large disordered aggregates. The large aggregates produced by the α-crystallin-Hsp 16.5 N-terminal construct suggest that specific interactions of the N-terminus with the core domain contribute to compact folding of the N-terminal region.

Packing of subunits into quaternary structures. Now that a partial structural picture of α-crystallin has been provided, the stage is set for an examination of some of the features that define its unique properties. Chief among them are its stability and its ability to form protein aggregates with micellar properties. Since most of its structure is held in common with sHSPs which behave differently (49), these features must correspond to limited regions and/or small details in the sequence. A limited number of regions can be identified that are of probable importance in this regard.

The N-terminal region of α-crystallin is significantly larger than the corresponding region in hsp 16.5. Good evidence suggests that the disordered 32 N terminal residues of Hsp 6.5 are packed inside the ‘hollow’ sphere formed by the 24 subunit aggregate. While it is likely that the corresponding N terminal regions of other sHSPs pack inside their aggregates, homology between these regions does not extend throughout the superfamily and ordered regions may be present in some cases. The interior ‘empty’ space, about 140,000 A⁰, is just large enough to accommodate these regions in Hsp 16.5, which are significantly more hydrophobic than those found on the outside of the sphere, leaving enough space for the packing of at most one additional domain (˜20,000 A⁰). If the N-terminal extension of α-crystallin is packed within the aggregate, it must prevent the formation of an ordered structure such as the 24 subunit spheroid of Hsp 16.5, because the larger hydrophobic region will not fit in such a small aggregate. As indicated by the dramatically altered properties of the α-crystallin_{Δ51 +} construct, removal of these residues is sufficient to produce soluble tetrameric α-crystallin. This supports the internal packing of these residues in the wild type aggregate, and suggests that hydrophobic interactions within the N-terminal region are important as a driving force in large aggregate formation.

The protein micelle model of crystallin aggregation has been successful in rationalizing many features of α-crystallin's behavior. It is instructive to briefly consider the characteristics of micelles formed by smaller amphipathic molecules; these characteristics are strongly affected by the relative sizes of the hydrophilic and hydrophobic regions. Amphipaths with small hydrophobic volumes and large hydrophilic cross sections form small aggregates because the hydrophilic region can tile the surface of a small sphere in which the hydrophobic volumes can pack. Amphipaths with larger hydrophobic volumes relative to hydrophilic cross section form larger aggregates so that the spherical surface tiled by the hydrophilic region contains a larger volume per subunit. For very large hydrophobic volumes or special geometrical constraints, other structures can be favored, ranging from non-spherical micelles to the familiar b-lamellar structures of phospholipid membranes. Our results suggest that the N-terminal region corresponds to the hydrophobic volume, while the hydrophilic cross section is provided by the common core domain.

Given the apparent packing of the N-terminal 32 residues of Hsp 16.5 in the interior of the aggregates, it is likely that the size and properties of the aggregates formed by members of the sHSP superfamily are in part controlled by the volume of the N-terminal extension. Without wishing to be bound by an particular theory, an important reason for N-terminal variability within the superfamily may be to control aggregate size, order, and geometry. This does not rule out the possibility that parts of this region are involved in more specific interactions with other monomers. The C-terminal extension is smaller, but could also have a role in interprotein interactions, particularly since the C-terminal region of the small heat shock protein already contains an unpaired β strand.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

All patents, applications, publications, test methods, literature, and other materials cited herein are hereby incorporated by reference.

What is claimed:

1. A truncated α-crystallin polypeptide derived from a wild-type α-crystallin protein, wherein said truncated polypeptide lacks an N-terminal sequence present in said wild-type protein.

2. The truncated α-crystallin polypeptide of claim 1 wherein said N-terminal sequence is hydrophobic.

3. The truncated α-crystallin polypeptide of claim 2 wherein said N-terminal sequence precedes a common domain in said wild-type protein.

4. The truncated α-crystallin polypeptide of claim 1 wherein said N-terminal sequence comprises residues 1-51 of said wild-type protein.

5. The truncated α-crystallin polypeptide of claim 4 comprising the sequence set forth in SEQ ID NO: 3.

6. An isolated polypeptide comprising an amino acid sequence encoded by a nucleic acid that hybridizes, under stringent conditions, to the complement of a nucleic acid encoding the polypeptide of claim 1.

7. An isolated polypeptide comprising an amino acid sequence encoded by a nucleic acid that hybridizes, under stringent conditions, to the complement of a nucleic acid encoding the polypeptide of claim 4.

8. The polypeptide of claim 1 which is at least 70% identical to a polypeptide comprising the amino acid sequence set forth in SEQ ID NO: 1.

9. The polypeptide of claim 1 which comprises an amino acid sequence at least 80% identical to a polypeptide comprising the amino acid sequence set forth in SEQ ID NO: 1 using a BLAST algorithm.

10. The polypeptide of claim 1 which comprises an amino acid sequence more than 90% identical to a polypeptide comprising the amino acid sequence set forth in SEQ ID NO: 1 using a BLAST algorithm.

11. The polypeptide of claim 1 further comprising a linker sequence at the N-terminus which is designed to enhance the solubility of said polypeptide.

12. An isolated nucleic acid encoding the truncated α-crystallin polypeptide of claim 1.

13. An isolated nucleic acid encoding the truncated α-crystallin polypeptide of claim 4.

14. An isolated nucleic acid that hybridizes, under stringent conditions, to the complement of a nucleic acid encoding the polypeptide of claim 1.

15. An isolated nucleic acid that hybridizes, under stringent conditions, to the complement of a nucleic acid encoding the polypeptide of claim 4.

16. The isolated nucleic acid of claim 12 that hybridizes, under stringent hybridization conditions, to the complement of a nucleic acid comprising the nucleotide sequence set forth in SEQ ID NO: 2 (FIG. 2).

17. The isolated nucleic acid of claim 15 that hybridizes, under stringent hybridization conditions, to the complement of a nucleic acid comprising the nucleotide sequence set forth in SEQ ID NO: 2 (FIG. 2).

18. An expression vector comprising:
(a) a nucleic acid encoding a small heat shock protein (sHSP); and
(b) a nucleic acid encoding a protein, polypeptide, or fragment thereof;
wherein said nucleic acids are operatively associated with an expression control sequence.

19. The expression vector of claim 18 wherein said sHSP is selected from the group consisting of a wild-type α-crystallin protein; a truncated α-crystallin polypeptide; thermophilic sHSP; a chimeric polypeptide comprising (a) a wild-type α-crystallin protein or a truncated α-crystallin polypeptide and (b) thermophilic sHSP; or combinations thereof.

20. The expression vector of claim 19 wherein said chimeric polypeptide comprises a truncated α-crystallin polypeptide and thermophilic sHSP.

21. The expression vector of claim 20 wherein said truncated α-crystallin polypeptide lacks an N-terminal sequence present in a wild-type α-crystallin protein.

22. The expression vector of claim 21 wherein said N-terminal sequence is hydrophobic.

23. The expression vector of claim 22 wherein said N-terminal sequence precedes a common domain in said wild-type protein.

24. The expression vector of claim 21 wherein said N-terminal sequence comprises residues 1-51 of said wild-type protein.

25. The expression vector of claim 21 comprising the sequence set forth in SEQ ID NO:2.

26. A method of enhancing expression of a protein in a host cell comprising coexpressing said protein with a small heat shock protein (sHSP).

27. The method of claim 26 wherein said sHSP is selected from the group consisting of a wild-type α-crystallin protein; a truncated α-crystallin polypeptide; a thermophilic sHSP; a chimeric polypeptide comprising (a) a wild-type α-crystallin protein or a truncated α-crystallin polypeptide and (b) a thermophilic sHSP; and combinations thereof.

28. The method of claim 27 wherein said chimeric polypeptide comprises a truncated α-crystallin polypeptide and a thermophilic sHSP.

29. The method of claim 28 wherein said truncated polypeptide lacks an N-terminal sequence present in a wild-type protein.

30. The method of claim 29 wherein said N-terminal sequence is hydrophobic.

31. The method of claim 30 wherein said N-terminal sequence precedes a common domain in said wild-type protein.

32. The method of claim 29 wherein said N-terminal sequence comprises residues 1-51 of said wild-type protein.

33. The method of claim 32 wherein said truncated polypeptide comprises the sequence set forth in SEQ ID NO: 3.

34. A thermotolerant host cell genetically modified to express a small heat shock protein.

35. The host cell of claim 34 wherein said sHSP is selected from the group consisting of a wild-type α-crystallin protein; a truncated α-crystallin polypeptide; a thermophilic sHSP; a chimeric polypeptide comprising (a) a wild-type α-crystallin protein or a truncated α-crystallin polypeptide and (b) a thermophilic sHSP; and combinations thereof.

36. The host cell of claim 35 wherein said chimeric polypeptide comprises a truncated α-crystallin polypeptide and a thermophilic sHSP.

37. The host cell of claim 36 wherein said truncated polypeptide lacks an N-terminal sequence present in said wild-type protein.

38. The host cell of claim 37 wherein said N-terminal sequence is hydrophobic.

39. The host cell of claim 37 wherein said N-terminal sequence precedes a common domain in said wild-type protein.

40. The host cell of claim 37 wherein said N-terminal sequence comprises residues 1-51 of said wild-type protein.

41. The host cell of claim 40 wherein said truncated polypeptide comprises the sequence set forth in SEQ ID NO: 3.


SEQUENCE LISTING (18 sequences)

SEQ ID NO: 1 - protein, 173 aa, Homo sapiens
  ref <308>: DATABASE ACCESSION NUMBER: GenBank / P02489
  ref <309>: DATABASE ENTRY DATE: 1986-07-21
  ref <313>: RELEVANT RESIDUES: (1)..(173)
MDVTIQHPWFKRTLGPFYPSRLFDQFFGEGLFEYDLLPFLSSTISPYYRQSLFRTVLDSGISEVRSDRDKFVIFLDVKHFSPEDLTVKVQDDFVEIHGKHNERQDDHGYISREFHRRYRLPSNVDQSALSCSLSADGMLTFCGPKIQTGLDATHAERAIPVSREEKPTSAPSS

SEQ ID NO: 2 - DNA, 372 bp, Homo sapiens
tccctcttccgcaccgtgctggactccggcatctctgaggttcgatccgaccgggacaagttcgtcatcttcctcgatgtgaagcacttctccccggaggacctcaccgtgaaggtgcaggacgactttgtggagatccacggaaagcacaacgagcgccaggacgaccacggctacatttcccgtgagttccaccgccgctaccgcctgccgtccaacgtggaccagtcggccctctcttgctccctgtctgccgatggcatgctgaccttctgtggccccaagatccagactggcctggatgccacccacgccgagcgagccatccccgtgtcgcgggaggagaagcccacctcggctccctcgtcctaa

SEQ ID NO: 3 - protein, 123 aa, Homo sapiens
SLFRTVLDSGISEVRSDRDKFVIFLDVKHFSPEDLTVKVQDDFVEIHGKHNERQDDHGYISREFHRRYRLPSNVDQSALSCSLSADGMLTFCGPKIQTGLDATHAERAIPVSREEKPTSAPSS

SEQ ID NO: 4 - protein, 147 aa, Methanocaldococcus jannaschii
MFGRDPFDSLFERMFKEFFATPMTGTTMIQSSTGIQISGKGFMPISIIEGDQHIKVIAWLPGVNKEDIILNAVGDTLEIRAKRSPLMITESERIIYSEIPEEEEIYRTIKLPATVKEENASAKFENGVLSVILPKAESSIKKGINIE

SEQ ID NO: 5 - protein, 150 aa, Oryza sativa
MSLVRRSNVFDPFSLDLWDPFDSVFRSVVPATSDNDTAAFANARIDWKETPESHVFKADLPGVKKEEVKVEVEEGNVLVISGQRSKEKEDKNDKWHRVERSSGQFMRRFRLPENAKVDQVKAGLENGVLTVTVPKAEVKKPEVKAIEISG

SEQ ID NO: 6 - protein, 158 aa, Pisum sativum
MSLIPSFFSGRRSNVFDPFSLDVWDPLKDFPFSNSSPSASFPRENPAFVSTRVDWKETPEAHVFKADLPGLKKEEVKVEVEDDRVLQISGERSVEKEDKNDEWHRVERSSGKFLRRFRLPENAKMDKVKASMENGVLTVTVPKEEIKKAEVKSIEISG

SEQ ID NO: 7 - protein, 145 aa, Caenorhabditis elegans
MSLYHYFRPAQRSVFGDLMRDMALMERQFAPVCRISPSESSEIVNNDQKFAINLNVSQFKPEDLKINLDGRTLSIQGEQELKTDHGYSKKSFSRVILLPEDVDVGAVASNLSEDGKLSIEAPKKEAVQGRSIPIQQAIVEEKSAE

SEQ ID NO: 8 - protein, 188 aa, Stigmatella aurantiaca
MADLSVRRGTGSTPQRTREWDPFQQMQELMNWDPFELANHPWFANRQGPPAFVPAFEVRETKEAYIFKADLPGVDEKDIEVTLTGDRVSVSGKREREKREESERFYAYERTFGSFSRAFTLPEGVDGDNVRADLKNGVLTLTLPKRPEVQPKRIQVASSGTEQKEHIKAYPAPAEPGLAAPLGWPGFS

SEQ ID NO: 9 - protein, 209 aa, Mus musculus
MTERRVPFSLLRSPSWEPFRDWYPAHSRLFDQAFGVPRLPDEWSQWFSAAGWPGYVRPLPAATAEGPAAVTLAAPAFSRALNRQLSSGVSEIRQTADRWRVSLDVNHFAPEELTVKTKEGVVEITGKHEERQDEHGYISRCFTRKYTLPPGVDPTLVSSSLSPEGTLTVEAPLPKAVTQSAEITIPVTFEARAQIGGPEAGKSEQSGAK

SEQ ID NO: 10 - protein, 173 aa, Bos taurus
MDIAIQHPWFKRTLGPFYPSRLFDQFFGEGLFEYDLLPFLSSTISPYYRQSLFRTVLDSGISEVRSDRDKFVIFLDVKHFSPEDLTVKVQEDFVEIHGKHNERQDDHGYISREFHRRYRLPSNVDQSALSCSLSADGMLTFSGPKIPSGVDAGHSERAIPVSREEKPSSAPSS

SEQ ID NO: 11 - protein, 175 aa, Bos taurus
MDIAIHHPWIRRPFFPFHSPSRLFDQFFGEHLLESDLFPASTSLSPFYLRPPSFLRAPSWIDTGLSEMRLEKDRFSVNLDVKHFSPEELKVKVLGDVIEVHGKHEERQDEHGFISREFHRKYRIPADVDPLAITSSLSSDGVLTVNGPRKQASGPERTIPITREEKPAVTAAPKK

SEQ ID NO: 12 - protein, 196 aa, Mus musculus
MDVTIQHPWFKRALGPFYPSRLFDQFFGEGLFEYDLLPFLSSTISPYYRQSLFRTVLDSGISELMTHMWFVMHQPHAGNPKNNPVKVRSDRDKFVIFLDVKHFSPEDLTVKVLEDFVEIHGKHNERQDDHGYISREFHRRYRLPSNVDQSALSCSLSADGMLTFSGPKVQSGLDAGHSERAIPVSREEKPSSAPSS

SEQ ID NO: 13 - DNA, 22 bp, Artificial Sequence
  oligonucleotide
tccctcttccgcaccgtgctgg

SEQ ID NO: 14 - DNA, 31 bp, Artificial Sequence
  oligonucleotide
gctttgttagcagctcgagccttaggacgag

SEQ ID NO: 15 - DNA, 48 bp, Artificial Sequence
  oligonucleotide
catatggacgtcaccaccggaaccggaaccaccggaaccaccgctagc

SEQ ID NO: 16 - DNA, 40 bp, Artificial Sequence
  oligonucleotide
ccagcacggtgcggaagagggagctagcggtggttccggt

SEQ ID NO: 17 - protein, 107 aa, Methanocaldococcus jannaschii
TGIQISGKGFMPISIIEGDQHIKVIAWLPGVNKEDIILNAVGDTLEIRAKRSPLMITESERIIYSEIPEEEEIYRTIKLPATVKEENASAKFENGVLSVILPKAESS

SEQ ID NO: 18 - protein, 105 aa, Bos taurus
SPYYRQSLFRTVLDSGISEVRSDRDKFVIFLDVKHFSPEDLTVKVQEDFVEIHGKHNERQDDHGYISREFHRRYRLPSNVDQSALSCSLSADGMLTFSGPKIPSG